SEQUENCE LISTING (96 sequences)

SEQ ID NO: 1 - DNA, 167 bp, SARS2 virus
tccagatgaggatgaagaagaagtctgaacaactggtgtaagagagcagcagaagtggcacaggtgattgtgaagaagaagagtcaacctgaagaagagcaagaactgattgtcctcactgccctcatattgagttgatggctcaacaaactgttggtcaacaagac

SEQ ID NO: 2 - DNA, 166 bp, SARS2 virus
tggctactaccgaagagcttgcagcattgttagcaggattctggcccagttcctaggtagtccagacgaattcgtggtggagacggcatcatatgggttgcacgggtgccaatgtgatctggactgagatctttcattttaccgtactgagggagccttgaataca

SEQ ID NO: 3 - DNA, 161 bp, Homo sapiens
ttgatgagctggagccacaccctcaatgcagagtcgtgtgaccctgaagactcggttttagccactgactcggatccctccgtgatatggctcttcgtttttttcttacatggctctggtcatgtggatggctgagttgttcatgctgagtactggacctc

SEQ ID NO: 4 - DNA, 17 bp, Artificial
  RNaseP - F3
ttgatgagctggagcca

SEQ ID NO: 5 - DNA, 18 bp, Artificial
  RNaseP - B3
caccctcaatgcagagtc

SEQ ID NO: 6 - DNA, 20 bp, Artificial
  RNaseP - Loop F
atgtggatggctgagttgtt

SEQ ID NO: 7 - DNA, 20 bp, Artificial
  RNaseP - Loop B
catgctgagtactggacctc

SEQ ID NO: 8 - DNA, 41 bp, Artificial
  RNaseP - FIP
gtgtgaccctgaagactcggttttagccactgactcggatc

SEQ ID NO: 9 - DNA, 45 bp, Artificial
  RNaseP - BIP
cctccgtgatatggctcttcgtttttttcttacatggctctggtc

SEQ ID NO: 10 - DNA, 18 bp, Artificial
  As1e - F3
cggtggacaaattgtcac

SEQ ID NO: 11 - DNA, 22 bp, Artificial
  As1e - B3
cttctctggatttaacacactt

SEQ ID NO: 12 - DNA, 28 bp, Artificial
  As1e - Loop F
ttacaagcttaaagaatgtctgaacact

SEQ ID NO: 13 - DNA, 27 bp, Artificial
  As1e - Loop B
ttgaatttaggtgaaacatttgtcacg

SEQ ID NO: 14 - DNA, 51 bp, Artificial
  As1e - FIP
tcagcacacaaagccaaaaatttatttttctgtgcaaaggaaattaaggag

SEQ ID NO: 15 - DNA, 49 bp, Artificial
  As1e - BIP
tattggtggagctaaacttaaagccttttctgtacaatccctttgagtg

SEQ ID NO: 16 - DNA, 18 bp, Artificial
  N2 - F3
cggcagtcaagcctcttc

SEQ ID NO: 17 - DNA, 20 bp, Artificial
  N2 - B3
ttgctctcaagctggttcaa

SEQ ID NO: 18 - DNA, 20 bp, Artificial
  N2 - Loop B
atggcggtgatgctgctctt

SEQ ID NO: 19 - DNA, 40 bp, Artificial
  N2 - FIP
tcccctactgctgcctggagcgttcctcatcacgtagtcg

SEQ ID NO: 20 - DNA, 42 bp, Artificial
  N2 - BIP
tctcctgctagaatggctggcatctgtcaagcagcagcaaag

SEQ ID NO: 21 - DNA, 20 bp, Artificial
  ORF1e - F3
ggctaactaacatctttggc

SEQ ID NO: 22 - DNA, 16 bp, Artificial
  ORF1e - B3
gtcagcacaaagccaa

SEQ ID NO: 23 - DNA, 20 bp, Artificial
  ORF1e - Loop F
tcttcaagccaatcaaggac

SEQ ID NO: 24 - DNA, 19 bp, Artificial
  ORF1e - Loop B
ttgtcggtggacaaattgt

SEQ ID NO: 25 - DNA, 51 bp, Artificial
  ORF1e - FIP
tctctaagaaactctacaccttcctttttactgtttatgaaaaactaaacc

SEQ ID NO: 26 - DNA, 23 bp, Artificial
  TATCTCAACCTGTGCTTGTGAAATTTTAGAATGTCTGAACACTCTC CT
tatctcaacctgtgcttgtgaaa

SEQ ID NO: 27 - DNA, 20 bp, Artificial
  IFIT2
ctgccgaacagctgagaatt

SEQ ID NO: 28 - DNA, 19 bp, Artificial
  IFIH1
tcagaaagcaatgcagaga

SEQ ID NO: 29 - DNA, 19 bp, Artificial
  OAS2
ctgagttctggctccacac

SEQ ID NO: 30 - DNA, 24 bp, Artificial
  CXCL10
ccaaggtctttagaaaaacttgaa

SEQ ID NO: 31 - DNA, 18 bp, Artificial
  MxA
agagtggctgtgggcaat

SEQ ID NO: 32 - DNA, 20 bp, Artificial
  IFITM2
agcctcccaactacgagatg

SEQ ID NO: 33 - DNA, 20 bp, Artificial
  PARP12
acgctgcctttctactttgt

SEQ ID NO: 34 - DNA, 21 bp, Artificial
  IFIT3
acagcagagacacagagggca

SEQ ID NO: 35 - DNA, 18 bp, Artificial
  MxB
ggaggcactgtcaggagt

SEQ ID NO: 36 - DNA, 18 bp, Artificial
  IRF9
gctgtctggaagactcgc

SEQ ID NO: 37 - DNA, 22 bp, Artificial
  RTP4
gagggaaaaaatggttgtagat

SEQ ID NO: 38 - DNA, 20 bp, Artificial
  IFI27
gccacggaattaacccgagc

SEQ ID NO: 39 - DNA, 19 bp, Artificial
  IFIT2
gccagtaggttgcacattg

SEQ ID NO: 40 - DNA, 18 bp, Artificial
  IFIH1
acttccttctgccaaact

SEQ ID NO: 41 - DNA, 20 bp, Artificial
  OAS2
ccctttggcttcagtttcct

SEQ ID NO: 42 - DNA, 18 bp, Artificial
  CXCL10
cttggaagcactgcatcg

SEQ ID NO: 43 - DNA, 19 bp, Artificial
  MxA
accagatcaggcttcgtca

SEQ ID NO: 44 - DNA, 18 bp, Artificial
  IFITM2
tcacgtcgccaaccatct

SEQ ID NO: 45 - DNA, 18 bp, Artificial
  PARP12
ccccttgccgtaggaagt

SEQ ID NO: 46 - DNA, 18 bp, Artificial
  IFIT3
tgtaggccaacaagttgt

SEQ ID NO: 47 - DNA, 20 bp, Artificial
  MxB
tgatgagctcatggctgatg

SEQ ID NO: 48 - DNA, 20 bp, Artificial
  IRF9
ccttcctctcagaggacaca

SEQ ID NO: 49 - DNA, 21 bp, Artificial
  RTP4
caacttcgctggcaggaggaa

SEQ ID NO: 50 - DNA, 18 bp, Artificial
  IFI27
ctgctatggaggacgagg

SEQ ID NO: 51 - DNA, 42 bp, Artificial
  IFIT2
tcaagttccaggtgaaatggcactgcaaccatgagtgagaac

SEQ ID NO: 52 - DNA, 46 bp, Artificial
  IFIH1
ccagatttggctgaactgtggtagaatttatcacaagttgatggtc

SEQ ID NO: 53 - DNA, 38 bp, Artificial
  OAS2
aactctcctcccgggaggtccagccccgaggtttatgc

SEQ ID NO: 54 - DNA, 44 bp, Artificial
  CXCL10
tctcttctcacccttctttttcattcctgcaagccaattttgtc

SEQ ID NO: 55 - DNA, 41 bp, Artificial
  MxA
gaccaccaccaggctgattgtcgcctgctgacattgggtat

SEQ ID NO: 56 - DNA, 39 bp, Homo sapiens
ggatcacggtggacatcgggtcaaggaggagcaggaagt

SEQ ID NO: 57 - DNA, 41 bp, Artificial
  PARP12
tccgttctgcttctgcatctgtagtacagaacctggccctc

SEQ ID NO: 58 - DNA, 44 bp, Artificial
  IFIT3
gttccaggtgaaatggcatttcaggtgaggtcaccaagaattcc

SEQ ID NO: 59 - DNA, 40 bp, Artificial
  MxB
gcctcacagggctgctttttcaagaggcagcggaatcgta

SEQ ID NO: 60 - DNA, 38 bp, Artificial
  IRF9
caacatccatgcggcccctcctgcgctgtgcactcaac

SEQ ID NO: 61 - DNA, 41 bp, Artificial
  RTP4
catccaacttcagcgtccatgctggacttgggagcagacat

SEQ ID NO: 62 - DNA, 38 bp, Artificial
  IFI27
caaaactacggcagagccagtctcacctcatcagcagt

SEQ ID NO: 63 - DNA, 42 bp, Artificial
  IFIT2
ggagggagaaaactccttggattggctttgaattcacgattc

SEQ ID NO: 64 - DNA, 42 bp, Artificial
  IFIH1
aggtctggggcatggagaattgtgtctgattctgaaactaca

SEQ ID NO: 65 - DNA, 41 bp, Artificial
  OAS2
acctgtttcacagtcctgcagcgtgcttcaccaggcgaatt

SEQ ID NO: 66 - DNA, 43 bp, Artificial
  CXCL10
gaatccagaatcgaaggccatcaccctctggttttaaggagat

SEQ ID NO: 67 - DNA, 41 bp, Artificial
  MxA
aatgtggacatcgccaccacaagattccgatggtcctgtct

SEQ ID NO: 68 - DNA, 41 bp, Artificial
  PARP12
ccatgtggtctggtccctgttcttcacggagtacgcgaatg

SEQ ID NO: 69 - DNA, 39 bp, Artificial
  PARP12
cggcaccagcgccatttttgcatgaacaccacagacccg

SEQ ID NO: 70 - DNA, 44 bp, Artificial
  IFIT3
gacagtgtctcaagggatctagaattgtagctttgaactcagtg

SEQ ID NO: 71 - DNA, 40 bp, Artificial
  MxB
cagctaccggaacaccgagcgacgttctgggctttgtgta

SEQ ID NO: 72 - DNA, 42 bp, Artificial
  IRF9
aggtgtatcagttgctgccacctgctgtcgctttgatggtac

SEQ ID NO: 73 - DNA, 40 bp, Artificial
  RTP4
aaccttcagctagactgcctaaccagccaaatgctctctg

SEQ ID NO: 74 - DNA, 40 bp, Artificial
  IFI27
tacagttgtgattggaggagttgattcccgccgcagtgaa

SEQ ID NO: 75 - DNA, 20 bp, Artificial
  IFIT2
gccgtaggctgctctccaag

SEQ ID NO: 76 - DNA, 23 bp, Artificial
  IFIH1
aagaagttgctcttccacttgag

SEQ ID NO: 77 - DNA, 25 bp, Artificial
  OAS2
ggatttatacagatcaatgagccct

SEQ ID NO: 78 - DNA, 25 bp, Artificial
  MxA
ggatgtacttcttgatgagtgtctt

SEQ ID NO: 79 - DNA, 17 bp, Artificial
  IFITM2
agcagggttgtggggca

SEQ ID NO: 80 - DNA, 23 bp, Artificial
  PARP12
cctttttgccactggtagacttc

SEQ ID NO: 81 - DNA, 19 bp, Artificial
  IFIT3
tgtggaaggattttctcca

SEQ ID NO: 82 - DNA, 17 bp, Artificial
  MxB
cacaggcgaccacgact

SEQ ID NO: 83 - DNA, 25 bp, Artificial
  IRF9
ggaacctccttaaattcagaactct

SEQ ID NO: 84 - DNA, 25 bp, Artificial
  RTP4
gttttgcctcttggattagttcttg

SEQ ID NO: 85 - DNA, 19 bp, Artificial
  IFI27
gccaccctgaccactttgg

SEQ ID NO: 86 - DNA, 25 bp, Artificial
  IFIT2
gacaaagtattttaccggactgagt

SEQ ID NO: 87 - DNA, 19 bp, Artificial
  OAS2
ctcccggcccaccaaacta

SEQ ID NO: 88 - DNA, 25 bp, Artificial
  CXCL10
tactgaaagcagttagcaaggaaag

SEQ ID NO: 89 - DNA, 20 bp, Artificial
  MxA
tctcagcatggcccaggagg

SEQ ID NO: 90 - DNA, 24 bp, Artificial
  IFITM2
caccctcttcatgaacacctgctg

SEQ ID NO: 91 - DNA, 21 bp, Artificial
  PARP12
cgccatctgccagcagaactt

SEQ ID NO: 92 - DNA, 22 bp, Artificial
  IFIT3
gatagagtgtgtaaccagattg

SEQ ID NO: 93 - DNA, 19 bp, Artificial
  MxB
tggccaggtggagaaagag

SEQ ID NO: 94 - DNA, 19 bp, Artificial
  IRF9
gccagccagggactcagaa

SEQ ID NO: 95 - DNA, 21 bp, Artificial
  RTP4
ggctcaagggtggaagcaata

SEQ ID NO: 96 - DNA, 17 bp, Artificial
  IFI27
tgctcagtgccatgggc

(19) United States
(12) Patent Application Publication
(10) Pub. No.: US 2026/0028685 A1
(43) Pub. Date: Jan. 29, 2026

(54) Saliva Stabilization Solution For Use In Nucleic Acid Amplification Reactions And Methods Of Use For The Detection Of Pathogen Nucleic Acids

(71) Applicant: The Regents of the University of Colorado, a body corporate, Denver, CO (US)

(72) Inventors: Sara L. Sawyer, Boulder, CO (US); Nicholas R. Meyerson, Broomfield, CO (US); Qing Yang, Longmont, CO (US)

(21) Appl. No.: 17/926,099

(22) PCT Filed: May 18, 2021
(86) PCT No.: PCT/US21/33040

§ 371 Date: Nov. 17, 2022

Related U.S. Application Data
(60) Provisional application No. 63/026,437, filed on May 18, 2020.
(60) Provisional application No. 63/049,417, filed on Jul. 8, 2020.

(51) Int. Cl. C12Q 1/70; C12Q 1/6806; C12Q 1/6851; C12Q 1/6888

(52) U.S. Cl. CPC C12Q 1/701; C12Q 1/6806; C12Q 1/6851; C12Q 1/6888

(57) ABSTRACT

In one aspect, the inventive technology relates to improved systems, methods, and compositions for a novel saliva stabilization solution for use in nucleic acid amplification reactions, and in particular embodiment its use in the detection of pathogen nucleic acids, such as SARS-CoV-2 (COVID-19).

### CROSS-REFERENCE TO RELATED APPLICATIONS

This International PCT Application claims the benefit of and priority to U.S. Provisional Application No. 63/026,437 filed May 18, 2020, and U.S. Provisional Application No. 63/049,417 filed Jul. 8, 2020. The entire specifications and figures of the above-referenced applications are hereby incorporated, in their entirety by reference.

### STATEMENT OF FEDERALLY SPONSORED RESEARCH

This invention was made with government support under grant number DP1-DA-046108 awarded by the National Institutes of Health. The U.S. government has certain rights in the invention.

### SEQUENCE LISTING

The instant application contains a Sequence Listing which has been submitted electronically in ASCII format and is hereby incorporated by reference in its entirety. Said ASCII copy, created on Apr. 26, 2023, is named “90245-00503-Sequence-Listing-AF-Corrected” and is 17,234 bytes in size.

### TECHNICAL FIELD

The inventive technology relates to the field of molecular diagnostic testing of pathogens, and in particular improved systems, methods, and compositions for a novel saliva stabilization solution for use in nucleic acid amplification reactions, such as colorimetric isothermal amplification reactions.

### BACKGROUND

The use of human saliva as a biospecimen for the detection of infectious diseases provides an effective non-invasive sampling method. Coupled with isothermal amplification technologies, saliva-based diagnostics allow for the rapid detection of pathogen nucleic acids. Some isothermal amplification technologies, such as reverse transcription loop-mediated isothermal amplification (RT-LAMP), can be carried out with the use of pH-sensitive dyes that produce a colorimetric change when amplification occurs. However, human saliva is inherently acidic and contains a strong physiologic buffer. Together, those characteristics of saliva can lead to false positives or negatives depending on the pH dye being used. As a result, there is a long-felt need for novel advancements to colorimetric assays to enhance the stability of sample nucleic acids, while increasing selectivity and sensitivity.

Here, the present inventors describe a novel saliva stabilization solution and methods of use thereof. In one embodiment, the novel saliva stabilization solution of the invention can be added to saliva before being processed for use as a template in colorimetric isothermal amplification reactions. Additionally, we describe a heating method to release pathogen and host RNA from saliva so that it can be accessed by amplification enzymes.

### SUMMARY OF THE INVENTION

In one aspect, the inventive technology relates to improved systems, methods, and compositions for a novel saliva stabilization solution for use in nucleic acid amplification reactions, such as amplification reactions with pH-dependent readouts, which in one embodiment may include colorimetric isothermal amplification reactions, and in particular embodiment its use in the detection of pathogen nucleic acids, such as SARS-CoV-2 (COVID-19). Another aspect of the invention includes a novel saliva stabilization solution for use in a LAMP diagnostic test, and preferably a buffer configured for use with an unprocessed saliva biological sample provided directly by a subject that is infected with, been exposed to, or is at risk of infection through exposure to an infectious agent, such as a viral, bacterial, fungal or parasite.

In one preferred aspect, the inventive technology relates to a kit for an improved M-RT-LAMP test for the detection of SARS-CoV-2 coronavirus RNA in saliva sample as well as one or more RNA biomarkers of infection produced by the subject's innate immune system that may be present in a saliva sample, and preferably an unprocessed, or minimally processed saliva sample from a subject that is infected with, been exposed to, or is at risk of infection through exposure to COVID-19 coronavirus.

Another aspect of the invention includes an improved Reverse Transcription-Loop-Mediated Isothermal Amplification (RT-LAMP) diagnostic test for pathogens. In one preferred aspect, the inventive technology relates to novel systems, methods, and compositions for an improved RT-LAMP test for the detection of SARS-CoV-2 RNA in a saliva sample, and preferably an unprocessed or minimally processed saliva sample from a subject that is infected with, been exposed to, or is at risk of infection through exposure to COVID-19 coronavirus.

In one preferred aspect, the detection of one or more RNA biomarkers of infection may indicate that the subject is in the early stages of a SARS-CoV-2 infection and possibly pre- or asymptomatic, while the absence of such RNA biomarkers of infection may indicated that the subject is in the late stage of a SARS-CoV-2 infection and is in a recovery phase, as well as possibly having an asymptomatic infection. Such differential infection data could be used to identify subjects having early, as well as late and asymptomatic infections. This data could further be used to design and implement quarantine procedures as well as inform the epidemiological study of the spread of SARS-CoV-2 within populations. In another aspect, this kit may be configured to detect SARS-CoV-2 coronavirus RNA and host biomarkers of infection may be used as part of a CLIA (Clinical Laboratory Improvement Amendments) certified high-complexity laboratory which may include a mobile, point-of-care testing apparatus as generally described herein.

Additional aspects of the inventive technology will become apparent from the specification, figures and claims below.

### BRIEF DESCRIPTION OF THE FIGURES

FIG. 1A-B: Optimized strategy for controlling natural variability in saliva pH. Saliva samples from 96 different individuals are analyzed for the prevalence of natural acidity extreme enough to trigger the pink-to-yellow color change of phenol red even before isothermal amplification. Each saliva sample was combined 1:1 with water (left) or 2× saliva stabilization solution (right; Materials and methods) and heated at 95° C. for 10 min to liberate RNA from virions. Two microliters of each was then added to 18 μL RT-LAMP reaction mix (2× Colorimetric RT-LAMP Master Mix, RNase P primers, nuclease-free water). The pictures show tubes immediately after samples and master mix are combined, before any incubation steps are undertaken to commence isothermal amplification. With raw saliva, 7 of 96 tubes turned yellow at this step (highlighted in red boxes). These are false positives because no amplification reaction has occurred. None of these 96 saliva samples mixed with saliva stabilization solution turned the reaction tube prematurely yellow. (B) Here, we show the method used to identify the ideal pH of the saliva stabilization solution used in (A) and throughout this paper. We chose four normal and four acidic saliva samples and mixed each 1:1 with 2× saliva stabilization solution containing NaOH at various concentrations (final molarity of NaOH after mixing shown). Samples were then heated at 95° C. for 10 min and combined with RT-LAMP reaction mix and control primers recognizing the human RNase P transcript. Before incubation, all tubes should be pink, and after incubation, all tubes should be yellow. Based on this, the red box indicates the final preferred NaOH concentration chosen.

FIG. 2A-B: Optimized RT-LAMP primer sets for detecting SARS-CoV-2 in human saliva. (A) Three RT-LAMP primer sets targeting the SARS-CoV-2 genome (AS1E [Rabe and Cepko, 2020], ORF1e, and CU-N2) were tested with real-time RT-LAMP. Saliva was mixed 1:1 with 2× saliva stabilization solution, heated at 95° C. for 10 min, and then spiked with in vitro transcribed SARS-CoV-2 RNA at the indicated concentrations. 4 μL of this was added to a master mix containing primers and NEB's WarmStart LAMP 2× Master Mix in a final reaction volume of 20 μL. Reactions were incubated at 65° C. and a fluorescence reading was taken every 30 s. EvaGreen was used to monitor amplification products in real-time (X-axis) using a QuantStudio3 quantitative PCR machine. There are nine lines for each of the three primer sets because three concentrations of spiked in SARS-CoV-2 RNA were each tested in triplicate (0, 400, 800 copies/μL saliva). When concentrations are given herein, denominator refers to the raw, pre-diluted saliva sample. The normalized change in fluorescence signal (ΔRn) is shown on the Y-axis. (B) Saliva mixed 1:1 with 2× saliva stabilization solution was heated (95° C. for 10 min) and then spiked with SARS-CoV-2 RNA at the indicated concentrations. Replicates were tested by RT-LAMP with the control RNaseP primer set and three distinct SARS-CoV-2 primer sets (AS1E, ORF1e, and CU-N2). All samples scored positive as expected except those boxed, which are saliva samples that contain no SARS-CoV-2 RNA.

FIG. 3A-B: The test limit of detection is 200 virions/μL. (A) Saliva samples were spiked with the indicated concentrations of heat-inactivated SARS-CoV-2 virions (top) before being diluted 1:1 with 2× saliva stabilization solution. Samples were then heated at 95° C. for 10 min and subjected to RT-LAMP at 65° C. for 30 min in six replicates. Each panel represents a unique primer set (listed at the bottom of each panel). The table shows a summary of positive reactions (yellow). Red box indicates the determined RT-LAMP limit of detection (LOD). (B) Saliva samples were spiked with heat-inactivated SARS-CoV-2 virions at 200 virions/μL (the limit of detection) before being diluted 1:1 with 2× saliva stabilization solution. Samples were then heated at 95° C. for 10 min and 20 replicates of RT-LAMP with the indicated primer sets were incubated at 65° C. for 30 min. The table shows a summary of positive reactions (yellow). Red box indicates primer sets to advance to subsequent analyses.

FIG. 4A-C: Evaluation of RT-LAMP on SARS-CoV-2-positive saliva samples from individuals with no reported symptoms at the time of sample collection. (A) We re-analyzed university saliva samples that had been previously analyzed for SARS-CoV-2 using quantitative RT-PCR with a primer set against the N gene of SARS-CoV-2 (see Materials and methods). The remaining saliva was mixed 1.1 with 2× saliva stabilization solution (without Proteinase K) and re-tested using RT-LAMP. The results of RT-LAMP are compared to relative saliva viral load determined by quantitative RT-PCR. The figure shows the distribution of the viral load of all 278 positive saliva samples separated by the corresponding RT-LAMP reaction results with either the AS1E or CU-N2 primer set. (B) Saliva RT-LAMP test sensitivity as a function of the cycle threshold (Ct) from the quantitative RT-PCR analysis. (C) A summary of test sensitivity and specificity from the blinded virion spike-in evaluation described above (FIG. 3—figure supplement 2) and from the human sample data in (A).

FIG. 5A-B: Assessment of Saliva TwoStep against a nasal swab test. (A) Matched nasal swabs and saliva from 54 individuals were analyzed (all of whom were SARS-CoV-2 positive at the time that these samples were collected, as verified by the saliva quantitative RT-PCR test described above). Nasal swab samples were collected within 2 days of positive saliva test and tested using the Quidel Direct Lyra RT-PCR test. The saliva samples from those same individuals were also re-tested with the Saliva TwoStep test. Data points represent individuals (n=54), and the corresponding test result is color-coded: positive, yellow; negative, gray. (B) Positive test agreement between Saliva TwoStep and the two comparator tests. The nature of the sample used by each test (nasal swab or saliva), and the test chemistry (quantitative RT-PCR or RT-LAMP) are delineated.

FIG. 6: Two step detection of SARS-CoV-2 in saliva. Step 1: Prepare saliva. Person provides 1 mL of saliva, and 1 mL of 2× saliva stabilization solution is then added to it. (This sample can be processed immediately or stored in the refrigerator at 4° C. for at least 4 days.) The mixture is heated at 95° C. for 10 min. This step serves to increase the pH of saliva, liberate viral RNA from virions in the saliva, and inactivate virions for safe handling (although appropriate safety precautions should always be taken). We have determined that performing a heating step at 95° C. for 30 min in a water bath before addition of the saliva stabilization solution also works well. However, in this case, Proteinase K must be left out of that solution. (Lower half) Step 2. Detect virus. Two microliters of stabilized saliva from step 1 is pipetted into each of three test tubes pre-filled with the RT-LAMP master mix and primers. The only thing different between the three tubes is the primer set included, with each set targeting either the human positive control RNA or a region of SARS-CoV-2 RNA, as indicated. After incubation, the reaction will turn from pink to yellow if the target RNA is present in saliva. An example of a positive and a negative test is shown.

FIG. 7A-B: Optimized stabilization buffer allows processing of acidic saliva samples while also maintaining color change of the RT-LAMP reaction. (A) Various final concentrations of NaOH were tested in the stabilization buffer to optimize sample collection over a range of saliva acidity. Red box indicates final optimum concentration after 1:1 mixture of saliva with stabilization buffer. Samples were boiled at 95° C. for 10 minutes and then analyzed in an RT-LAMP reaction by incubating at 65° C. for 30 minutes. All reactions contained a primer set targeting the human RNaseP transcript. (B) Optimized buffer was used to confirm no effect on limit of detection of SARS2 in vitro transcripts in RT-LAMP. Four or two replicates were analyzed at the indicated amount of SARS2 RNA spike-in. Each replicate was tested with RNaseP primer set and three distinct SARS2 primer sets (As1e, ORF1e, and N2).

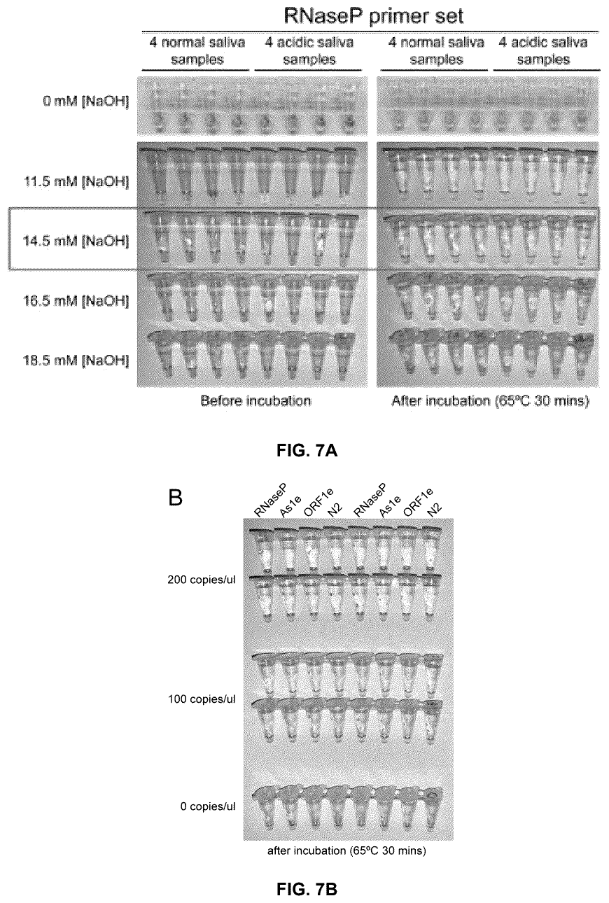

FIG. 8A-C: Optimized heat inactivation for safely detecting SARS-CoV-2 in human saliva. (A) This experiment shows that heating at 95° C. for 10 min degrades viral RNA when it is not in the form of virions. Saliva samples were diluted 1:1 with saliva stabilization solution. In vitro transcribed SARS-CoV-2 RNA was spiked into the diluted saliva to reach the indicated concentrations before (left) or after (right) the heating at 95° C. for 10 min. To match other experiments, the indicated concentration represents the copies of SARS-CoV-2 RNA in the original undiluted saliva. The samples were then subjected to RT-LAMP at 65° C. for 30 min. In this colorimetric version of RT-LAMP, reactions remain pink when no amplification occurred, and turned yellow if there was an amplification event. An RT-LAMP primer set targeting the human RNaseP transcript is included as a host RNA amplification control in addition to the three SARS-CoV-2 primer sets shown in (A). (B) This experiment shows that heating saliva at 95° C. for 10 min does not degrades viral RNA when it is in the form of virions. Saliva samples were spiked with the indicated concentrations of heat-inactivated SARS-CoV-2 virions before being diluted 1:1 with saliva stabilization solution. Samples were then heated at 95° C. for 10 min and subjected to RT-LAMP similarly to the experiment shown in (A). (C) Results illustrate the preferred incubation time at 95° C. to liberate SARS-CoV-2 RNA from virions. Saliva samples were spiked with the indicated concentrations of heat-inactivated SARS-CoV-2 virions before being diluted 1:1 with saliva stabilization solution. Samples were then heated at 95° C. for the indicated amount of time and subjected to RT-LAMP similarly to the experiment shown in (B). Without heating, no SARS-CoV-2 RNA can be detected with RT-LAMP, presumably because virions remain intact and the viral RNA is not accessible by the amplification enzymes. Amplification is somewhat inconsistent at 5 and 30 min possibly because at 5 min hardly any RNA has been liberated, and by 30 min, it has been largely degraded. However, 10 or 15 min at 95° C. appears to provide just the right balance between liberating and preserving RNA. All reactions contain the AS1E primer set. Duplicates are presented at each time point.

FIG. 9A-B: Saliva samples are stable at 4° C. for at least 4 days before processing, if stored in saliva stabilization solution. (A) Schematic of the experimental conditions. (B) RT-LAMP reaction result before and after the isothermal amplification. Saliva samples were spiked with heat-inactivated SARS-CoV-2 virions at the indicated concentration and mixed 1:1 with saliva stabilization solution or nuclease-free water before/after storing at 4° C. for 24, 48, 72, and 96 hr. Samples were then heated at 95° C. for 10 min and analyzed using RT-LAMP with the indicated primer sets. Condition C, which is the condition used in our test, performs robustly and is sensitive to the limit of detection even after 96 hr storage at 4° C. The stated limit of detection of 200 virions/μL is boxed.

FIG. 10A-B: Saliva TwoStep primers will detect most or all currently circulating viral variants of concern. (A) Genome map of SARS-CoV-2 with the regions targeted RT-LAMP primers highlighted in red. SARS-CoV-2 genome map is adapted from BioRender. (B) Sequence alignments of regions of the key SARS-CoV-2 genome variants targeted by RT-LAMP primer sets AS1E and CU-N2. Binding regions of each individual primer set component is highlighted in underlying horizontal bars. The SARS-CoV-2 genome region targeted by AS1E primer set is conserved among all variants. For CU-N2, the red box highlights region of sequence variation that might render CU-N2 primer set less effective to identify the UK and Brazil variants. The coordinate of the genome sequence is based on the SARS-CoV-2 reference genome (NCBI NC_045512.2). The SARS-CoV-2 variant representative genomes are downloaded from GISAID (South Africa Variant B.1.351: hCoV-19/South Africa/KRISP-EC-K004572/2020; UK Variant B.1.1.7: hCoV-19/England/MILK-9E2FE0/2020; Brazil Variant P.1: hCoV-19/USA/VA-DCLS-2185/2020).

FIG. 11A-B: Saliva stabilization solution containing NaOH does not lower sensitivity of colorimetric RT-LAMP detection of SARS-CoV-2. (A) Here, the detection limit of the RT-LAMP assay was assessed in the absence of any saliva or saliva stabilization solution. This was explored in order to determine whether there might be components of saliva or saliva stabilization solution that inherently lower test sensitivity because they are inhibitory to the RT-LAMP reaction. Here, synthetic SARS-CoV-2 RNA was diluted in nuclease-free water. The diluted RNA was mixed with RT-LAMP reaction mix and incubated at 65° C. for 30 min to allow isothermal amplification. Positive reactions turn yellow. Two different primer sets that amplify SARS-CoV-2 were employed, AS1E and CU-N2. The red box indicates the concentration at which positives were identified at least 95% of the time (here, 100% is achieved). That is defined at the limit of detection. Here, it is 200 copies/μL, just as when saliva and saliva stabilization solution is used (see panel B, and data figures in main paper). (B) Evaluation of RT-LAMP detection limit in the presence of saliva, but in the presence or absence of saliva stabilization solution. Saliva spiked with heat-inactivated SARS-CoV-2 virions at specified concentrations was mixed 1:1 with stabilization solution (left) or nuclease-free water (right) and heated at 95° C. for 10 min (RNA liberation) before being incubated at 65° C. for 30 min (isothermal amplification). On the left, the saliva stabilization solution achieves a limit of detection of 200 virions/μL. When virions are boiled without the saliva stabilization solution (right), very few reactions turn positive and the pattern is unpredictable, presumably because virions and viral RNA are destroyed.

FIG. 12A-B: Blinded sample evaluation. Plain saliva, or saliva spiked with heat-inactivated SARS-CoV-2 virions at different concentrations, was heated at 95° C. for 10 minutes. Samples were then analyzed using RT-LAMP by a researcher that did not know the true state of each sample. Experiments in figure) For each sample, three reactions were performed as indicated by each triplet of tubes. By looking at the patterns of yellow and pink results in each triplet, samples were scored according to the criteria in table A. The true status and observed result of each sample are listed to the right in green boxes (P=Positive, N=Negative, I=Inconclusive). A white box on the triplet is shown if the sample contained SARS-CoV-2. One sample resulted in inconclusive test result. This sample did have SARS-CoV-2 spiked into it, but one of the SARS-CoV-2 primer sets failed to produce a signal (CU-N2). This failed reaction is still pink (negative) even though the tube has 2×LOD virus. 1×LOD=200 virions/μL. Summary statistics for this experiment are provided in tables B and C below.

FIG. 13: Quantitative RT-PCR standard curve used to determine the Ct value to virion/μL calculation. 10,000 copies/μL of heat deactivated SARS-CoV-2 virus was spiked into negative saliva specimens from six different volunteers and incubated for 30 min at 95° C. Samples were diluted to indicate concentrations using heat-treated saliva without SARS-CoV-2 addition from the same individual. The standard curve for the primer set targeting SARS-CoV-2 N gene is generated from the linear regression analysis and is illustrated with 95% confidence interval.

FIG. 14: Near normal distribution of quantitative RT-PCR raw Ct values of SARS-CoV-2 N gene from positive individuals. Between Sep. 16-25, 2020, 8836 saliva samples were screened for SARS-CoV-2 using the direct quantitative RT-PCR method. (A) The amplification of SARS-CoV-2 N gene is detected in 347 samples and the Ct value distribution is illustrated. Samples with Ct values above the qRT-PCR limit of detection (Ct <34, dark gray, N=278) were considered SARS-CoV-2 positive and used for RT-LAMP validation. (B) Quantile—quantile plot of the SARS-CoV-2 N Ct values indicates near normal distribution within the linear range of qRT-PCR (D'Agostino test, K2=9.07, p-value=0.011).

FIG. 15: Shows the results of an exemplary SARS-CoV-2 RT-LAMP test showing a positive result for the detection of SARS2 RNA shown with the color change from red to yellow. The top row shows the positive detection of RNA oligos for the ORF1ab gene (SEQ ID NO. 1) of SARS-CoV-2. The bottom row shows the positive detection of RNA oligos for the N gene (SEQ ID NO. 2) of SARS-CoV-2. Water was used as the template in the first column of reactions (no SARS2) and a synthetic RNA oligo SARS2 standard was used in reactions in the second column (+SARS2 RNA).

### DETAILED DESCRIPTION OF THE INVENTION

In one embodiment, the present invention includes a saliva stabilization solution that may be used to stabilize nucleic acids, such as DNA and RNA, in amplification reactions with pH-dependent readouts, such as for example nucleic acid amplification reactions like colorimetric isothermal amplification reactions, an example being a RT-LAMP-assay. The saliva stabilization solution offers several significant advantages, namely: (1) it reduces the pH variability of human saliva thereby eliminating false positives, (2) it lowers the viscosity of saliva, and (3) stabilizes nucleic acids, such as RNA for analysis in colorimetric isothermal amplification reactions, such as a RT-LAMP test. As detailed below, the present inventors validated the saliva stabilization solution of the invention using an exemplary RT-LAMP test incorporating a large cohort of saliva and matched nasal swab specimens collected from a local university population, comparing the test to two other quantitative RT-PCR-based SARS-CoV-2 tests (one nasal test and one saliva test). The present inventors found that our optimized RT-LAMP procedure and solution performs consistently with high specificity and sensitivity, even though our samples were largely from individuals who had no reported symptoms at the time of sample collection.

The saliva stabilization buffer of the invention may be applied to a variety of colorimetric diagnostic tests and/or pH sensitive dyes incorporated therewith, an in particular isothermal nucleic acid amplification test using pH sensitive dyes such as strand-displacement amplification (SDA), helicase-dependent amplification (HDA), and loop-mediated isothermal amplification (LAMP), and polymerase-chain reaction (PCR). As noted elsewhere, LAMP in particular has been used in a number of field and point-of-care diagnostics. LAMP reactions use a strand-displacing DNA polymerase (and reverse transcriptase for RNA targets) with four to six primers, resulting in highly exponential amplification. This high degree of DNA synthesis facilitates visual detection of positive amplification based on the precipitation of magnesium pyrophosphate. Detection can be performed in real time with a specialized turbidity instrument or by direct visual assessment, although the former typically requires a long incubation time (≥60 min), and the precipitate can be difficult to see under even ideal conditions. Alternative detection methods for nucleic acid amplification use the color change of a metal-sensitive indicator, such as a shift from dark yellow to yellow (calcein), dark blue to blue (hydroxynaphthol blue), or dark blue to light blue (malachite green). Notably, the saliva stabilization solution of the invention may be used with pH indicator dyes, such as bromocresol purple, neutral red, phenol red, cresol red, naphtholphthalein, m-cresol purple, thymol blue, and phenolphthalein to provide better and more accurate results.

As noted below, in a preferred embodiment a reaction vessel may be pre-loaded or may accept a quantity of saliva stabilization solution. In this embodiment, the saliva stabilization solution may be specially configured to dilute the saliva such that it stabilizes any nucleic acids, such as DNA or RNA in the sample and conserves the pH-induced color change in the colorimetric reaction, for example as demonstrated in the figures in an exemplary RT-LAMP test. As noted above, unprocessed saliva is slightly acidic, such that adding saliva to the LAMP reaction may artificially change the color without a reaction taking place resulting in a false positive. In certain embodiments, the saliva stabilization solution of the invention may include: a quantity of a reducing agent, a quantity of a chelating agent, and a quantity of one or more base solutions. In one preferred embodiment, the saliva stabilization solution of the invention may include: a quantity of DDT as a reducing agent, a quantity of EDTA as a chelating agent, and a quantity of NaOH as a strong base. In this preferred embodiment, the saliva stabilization solution of the reaction vessel may include: between 100-300 mN DDT, 1-3 nM EDTA, and between 10-30 mM of NaOH, and more preferably between 100-300 mN DDT, 1-3 nM EDTA, and between 10-30 mM of NaOH.

In another embodiment a saliva stabilization solution may include: a quantity of TCEP; a quantity of EDTA; a quantity of NaOH; a quantity of Proteinase K; and a quantity of DEPC-treated water, and wherein said saliva stabilization solution is combined with a saliva sample at a 1:1 ratio. In one specific embodiment, a saliva stabilization solution may include: 5 mM TCEP, 2 mM EDTA, 29 mM NaOH, 100 μg/mL Proteinase K, diluted in DEPC-treated water, and wherein said saliva stabilization solution is combined with a saliva sample at a 1:1 ratio.

In another preferred embodiment, a saliva stabilization solution may be optimized to reduce false positives and preserve the color change of the LAMP assay. In this embodiment, the optimized saliva stabilization solution may include a concentration of NaOH buffer is between 11.6 and 16.4 mM, and preferably 14.5 mM in a typical buffer sample.

In additional embodiments, the invention provides for an enhanced saliva stabilization solution that can stabilize and preserve the saliva sample for an extended period of time at room temperature before the RT-LAMP reaction is allowed to proceed. In this embodiment, a saliva stabilization solution or buffer of the invention may further include: 1) a detergent that may help in cell lysis and the inhibition of RNase enzymes present in saliva; 2) a monovalent salt that may stabilize RNA at room temperature; and 3) a pH adjustor to modify the pH to enhance RNA stabilization and counteract pH fluctuations from other components. In a specific preferred embodiment, the enhanced saliva stabilization solution of the invention may include: 1) a detergent (0.1-10%), selected from the group consisting of SDS, Triton-X, NP-40, or Tween-20; 2) a monovalent salt (0.01-1 M), selected from the group consisting of lithium chloride or sodium chloride; 3) and a pH adjustor which may be a general acid or base compound.

As noted above, the invention may include a reaction vessel containing a saliva sample from a subject, wherein said a reaction mixture may comprise at least one RT-LAMP assay primer set specific for a pathogen, such as SARS-CoV-2, and preferably further comprising primers sets identified in Table 2 below, and optionally at least one assay primer set for one or more host RNA biomarkers of infection and preferably a host RNA biomarkers of infection selected from Table 7; at least one set of control primers; a saliva stabilization solution; a reverse transcriptase enzyme; RNase inhibitor, and other reagents necessary to produce an RT-LAMP reaction. The reaction mixture and saliva sample from a subject may be incubated to promote generation of RT-LAMP reaction products. In this preferred embodiment, the step of incubating the reaction mixture and saliva sample may include initially applying a high-heat to the said saliva sample and saliva stabilization solution, preferably to at least 95° C. for 10 minutes, followed by addition of the reaction mixture containing the components needed for the LAMP reaction. (As noted below, this step may be generally referred to as boiling the reaction mixture and saliva sample) This initial heating step may be followed by heating the reaction mixture and said saliva sample at a lower heat, preferably to at least at 65° C. for 30 minutes, after which the incubation may be stop by applying a higher heat to stop the RT-LAMP reaction, however not so high as to destroy the reaction products. This deactivating heating step may preferably include heating said reaction mixture and said saliva sample at least 80° C. for 5 minutes.

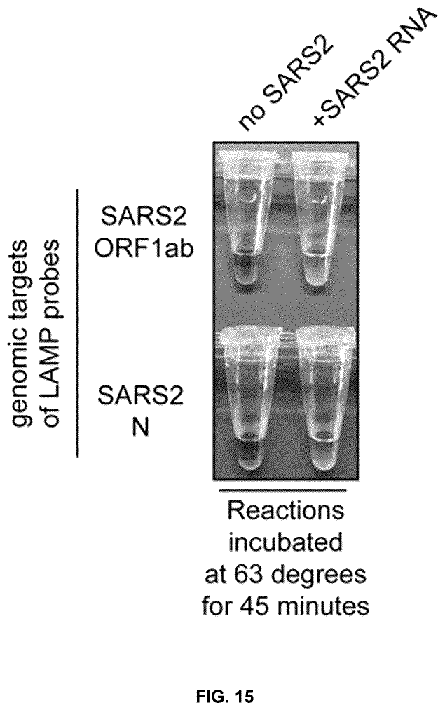

As noted, once the RT-LAMP reaction is allowed to proceed the contents of the reaction vessel may undergo visual color change (negative result is red positive result is yellow in the example shown in exemplary FIG. 15) due to a decrease in pH resulting from the amplification of target nucleic acids. Naturally, such visual color change is exemplary only as additional visual or other quantifiable signals may be contemplated within the invention's scope, such as UV fluorescence reactions and the like. In another embodiment, once the RT-LAMP reaction is allowed to proceed, the contents of the reaction vessel may undergo visual color change indicative or the presence of a target nucleic acid, such as an exemplary SARS-CoV-2 RNA transcript produced by the reaction, as well as optionally the presence of absence of host RNA biomarkers of infection. In this optional multivariate (M-RT-LAMP) test, the detection of host RNA biomarkers of infection, for example may be through a third color change or UV light signal indicating that that the pathogen, in this case being SARS-CoV-2 coronavirus, is in the early stages of infection, while a negative result for host RNA biomarkers of infection, coupled with a positive SARS-CoV-2 coronavirus result indicates that the subject is in the later stage or recovering from a SARS-CoV-2 coronavirus infection. The novel RNA-based M-RT-LAMP assay for the detection of SARS-CoV-2 and host biomarkers of infection in a saliva sample may be administered in one or more Clinical Laboratories that are Clinical Laboratory Improvement Amendments of 1988 (CLIA), 42 U.S.C. § 263a certified high-complexity laboratories.

In other embodiments, the invention includes systems, methods, and compositions for a novel diagnostic assay for the detection of SARS-CoV-2 nucleic acids from a biological sample, such as a respiratory sample (e.g., saliva, (bronchoalveolar lavage) BAL, nasopharyngeal (NP) swabs, etc. . . . ), and preferably from a saliva sample. Notably, SARS-CoV-2 RNA is generally detectable in respiratory specimens during the acute phase of infection.

In one preferred embodiment, invention includes systems, methods, and compositions for a novel RNA-based RT-LAMP assay for the detection of SARS-CoV-2 in an unprocessed, or minimally process saliva sample. In this embodiment, a saliva sample from a subject is placed into a reaction vessel, that may already contain a saliva stabilization solution and other components necessary to carry out the RT-LAMP reaction of the invention. Naturally, in alternative embodiments, saliva stabilization solution and other components may be added with, or after the deposit of the saliva sample into the reaction vessel by the subject.

Next, one or more target nucleic acid sequences from a target pathogen, such as an exemplary the genome of the SARS-CoV-2 coronavirus, to the extent present in the unprocessed, or minimally processed saliva sample may be amplified. In this embodiment, the isothermal amplification RT-LAMP reaction may occur within the reaction vessel and amplify the target sequences from the genome of SARS-CoV-2 producing a quantity of RNA transcripts. This RT-LAMP reaction may be allowed to proceed for between 20 minutes and 1 hour, and preferably between 30-45 minutes, and can preferably be subject to a heating element, such as a heat block, or other such similar device that may allow the RT-LAMP reaction to proceed at a controlled temperature. For example, in this embodiment, the RT-LAMP test of the assay may include one or more LAMP primer sets that specifically amplifies one or more target regions of the SARS-CoV-2 genome. In one preferred embodiment, SARS-CoV-2 may be selected from the primer sequences identified herein.

In alternative embodiments, the RT-LAMP test of the assay may include one or more LAMP primer sets that specifically amplifies the ORF1ab region of SARS-CoV-2 (SEQ ID NO. 1; see also Table 1). Exemplary RT-LAMP primers are listed in Table 1-2. Primer sets include an outer forward primer (F3), outer backward primer (B3), forward inner primer (FIP), backward inner primer (BIP), loop forward primer (LF), and loop backward primer (LB). Additional primer sets may include primers configured to specifically amplify gene N of SARS-CoV-2 region of SARS-CoV-2 (SEQ ID NO. 2; see also Table 2), as well as primers configured to amplify a host target gene as a control. In a preferred embodiment, this host target control gene may include RNaseP (SEQ ID NO. 3). As discussed below, similar primers directed to host RNA biomarkers of infection may further be identified in Table 10.

In alternative embodiment, the RT-LAMP test of the assay may include one or more LAMP primer sets that specifically amplifies the specific regions of SARS-CoV-2 genome, specifically identified herein as As1e, ORF1e and CU-N2. In one embodiment, the invention may include one or more assay primer set specific for SARS-CoV-2, further comprising primers sets As1e, ORF1e and CU-N2. In this embodiment, assay primer set As1e may include nucleotide sequences according to SEQ ID NO's. 10-15, and assay primer set N2 may include the nucleotide sequences according to SEQ ID NO's. 16-20, and ORF1e according to SEQ ID NO's. 21-26. As noted below, in one preferred embodiment, the RT-LAMP test of the assay may include one or more control primer sets that specifically amplifies a target host gene. In this preferred embodiment, control primers comprise primers for RNaseP, which may include the nucleotide sequences according to SEQ ID NO. 4-9.

In another preferred embodiment, invention includes systems, methods, and compositions for a novel Multivariate Reverse Transcription-Loop-Mediated Isothermal Amplification (M-RT-LAMP) assay for the detection of SARS-CoV-2 and one or more host RNA biomarkers of infection in a saliva sample. In this embodiment, a saliva sample from a subject is placed into a reaction vessel, that may already contain a saliva stabilization solution and other components necessary to carry out the M-RT-LAMP reaction of the invention. As noted above, primers directed to amplify target sequences from the genome of SARS-CoV-2 may be included in the reaction vessel. In a preferred embodiment, a plurality of SARS-CoV-2 primers may be used, for example SARS2-PrimerSet1, and 2 identified herein or primer sets for As1e and N2 and ORF1e respectively (SEQ ID NO's. 10-15, and SEQ ID NO's. 16-20, and SEQ ID NO's. 21-26), to ensure adequate amplicon coverage and detection of the virus.

In this preferred embodiment, one or more additional primers directed to host RNA biomarkers of infection, for example the primers according to the sequences identified in Table 5, may be included in the RT-LAMP reaction. Notably, such specific target RNA transcripts or biomarkers produced by a patient's innate immune response may be indicative of early infection. As generally described in U.S. Provisional Patent Nos. 63/006,570, and 63/006,561, (both of which being specifically incorporated herein in their entirety by reference), host RNA biomarkers in saliva may be at their highest level of expression just before symptoms appear in a subject, after which point their numbers begin to decline as other aspects of the host's immune system begin to drive the subject's infection response. As a result of this insight, the M-RT-LAMP test of the invention can not only identify whether a subject is positive for SARS-CoV-2, but also the presence, or lack thereof of host RNA biomarkers of infection that may allow the differentiation between pre-symptomatic from symptomatic subjects, as well as recovering individuals in the later stages of infection. In this manner, detection of host biomarkers of infection may act as a secondary indication of infection that when coupled with the detection of SARS-CoV-2 can identify not only the presence of the viral pathogen, but the stage of infection, as well as if the subject is symptomatic or asymptomatic. Such data can be collected and used to better inform quarantine protocols as well as therapeutic treatments and the like.

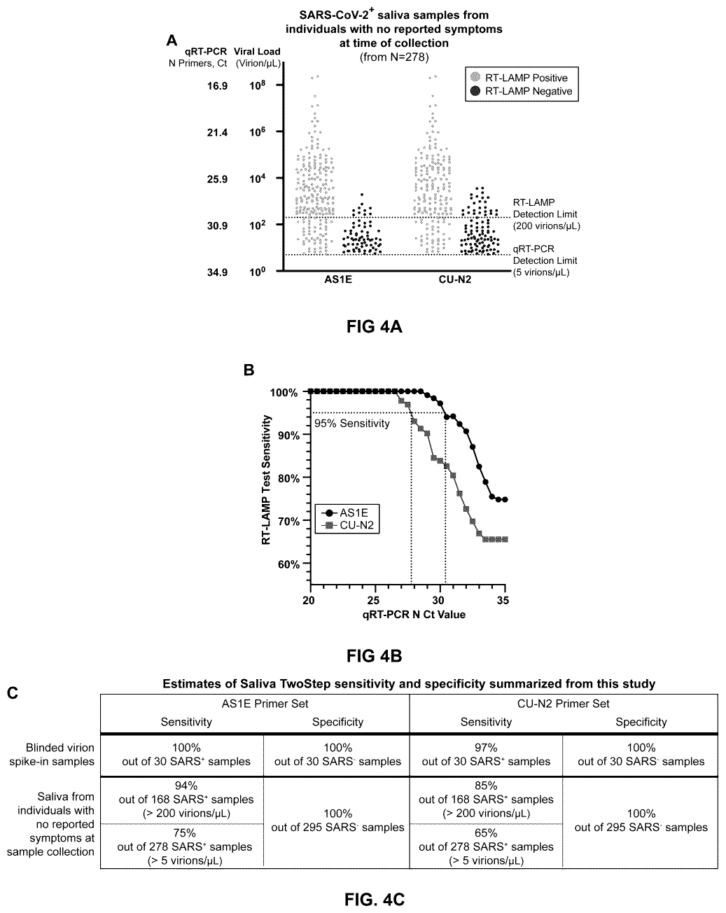

As a result, in one preferred embodiment, the isothermal amplification M-RT-LAMP reaction may occur within the reaction vessel and amplify the target sequences from the genome of SARS-CoV-2 producing a quantity of RNA transcripts, as well as one or more host RNA biomarkers of infection. Examples of host biomarkers of infection have been identified under the sequence listing section of U.S. Provisional Patent Application No. 63/006,570. (Nucleotide sequences according to SEQ ID NO. 1-468 are specifically incorporated herein by reference). The M-RT-LAMP reaction may be allowed to proceed for between 20 minutes and 1 hour, and preferably between 30-45 minutes, and can preferably be subject to a heating element, such as a heat block, or other such similar device that may allow the M-RT-LAMP reaction to proceed at a controlled temperature. As noted in FIG. 4, the M-RT-LAMP may identify if a patient is infected with SARS-CoV-2, as well as what stage the infection is at—for example early or late stage depending on the presence of host biomarkers of infection. Additionally, in the event the M-RT-LAMP returns a negative result for SARS-CoV-2, this could be indicative of either the early stages of a separate infection, or the early stages of a SARS-CoV-2 that was not detected by the M-RT-LAMP test. In this manner, the presence of host biomarkers of infection may provide a second layer of control to better inform the subject of their infection status. In such instances, it may be recommended that the subject quarantine and re-test to determine if the early host biomarkers of infection did, in fact, indicate an early SARS-CoV-2 infection.

According to this embodiment of the present invention, the present invention also provides a kit, in particular a kit of parts, comprising the components, reagents, and nucleotide primers that may be required to perform RNA-based M-RT-LAMP assay for the detection of SARS-CoV-2 and host RNA biomarkers of infection in a saliva sample. For example, in one embodiment a kit of the invention may include a saliva reaction vessel, a saliva stabilizing buffer, one or more nucleotide primers configured to amplify target genome sequences in the SARS-CoV-2 coronavirus, such as SEQ ID NO's. 10-20, nucleotide primers configured to amplify one or more host RNA biomarkers of infection, nucleotide primers configured to amplify one or more controls, such as SEQ ID NO's. 4-10, a temperature regulation element to cool or heat the sample, a centrifuge, a mixer to mix the sample such as a vortex machine and optionally technical instructions with information on how to perform and interpret the RNA-based M-RT-LAMP assay for the detection of SARS-CoV-2 and host biomarkers of infection in a saliva sample. Such kits, preferably kits of parts, may be applied e.g., for any of the above mentioned applications or uses, preferably for the use of performing the novel RNA-based M-RT-LAMP assay of the invention for the detection of SARS-CoV-2 and host biomarkers of infection in a saliva sample. Moreover, in another embodiment, the kit may additionally contain parts and/or devices necessary or suitable for the collection of saliva samples, as well as personal protective equipment such as facemasks, eye protection and protective clothing or gowns. Additional embodiments may include identification markers that may be associated with each saliva sample, for example, a barcode, a QR code, or other digitizable code configured to cross-reference a sample with a subject.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Nucleic acids and/or other moieties of the invention may be isolated or “extracted.” As used herein, “isolated” means separate from at least some of the components with which it is usually associated whether it is derived from a naturally occurring source or made synthetically, in whole or in part. Nucleic acids and/or other moieties of the invention may be purified. As used herein, purified means separate from the majority of other compounds or entities. A compound or moiety may be partially purified or substantially purified. Purity may be denoted by weight measure and may be determined using a variety of analytical techniques such as but not limited to mass spectrometry, HPLC, etc.

The term “primer,” as used herein, refers to an oligonucleotide capable of acting as a point of initiation of DNA synthesis under suitable conditions. Such conditions include those in which synthesis of a primer extension product complementary to a nucleic acid strand is induced in the presence of four different nucleoside triphosphates and an agent for extension (for example, a DNA polymerase or reverse transcriptase) in an appropriate buffer and at a suitable temperature.

A primer is preferably a single-stranded DNA. The appropriate length of a primer depends on the intended use of the primer but typically ranges from about 6 to about 225 nucleotides, including intermediate ranges, such as from 15 to 35 nucleotides, from 18 to 75 nucleotides and from 25 to 150 nucleotides. Short primer molecules generally require cooler temperatures to form sufficiently stable hybrid complexes with the template. A primer need not reflect the exact sequence of the template nucleic acid but must be sufficiently complementary to hybridize with the template. The design of suitable primers for the amplification of a given target sequence is well known in the art and described in the literature cited herein.

As used herein, a biological marker (“biomarker” or “marker”) is a characteristic that is objectively measured and evaluated as an indicator of normal biologic processes, pathogenic processes, or pharmacological responses to therapeutic interventions, consistent with NIH Biomarker Definitions Working Group (1998). Markers can also include patterns or ensembles of characteristics indicative of particular biological processes. The biomarker measurement can increase or decrease to indicate a particular biological event or process. In addition, if the biomarker measurement typically changes in the absence of a particular biological process, a constant measurement can indicate occurrence of that process. In a preferred embodiment a biomarker includes one or more RNA transcripts that may be indicative of infection or other normal or abnormal physiological process.

As referred to herein, the terms “nucleic acid”, “nucleic acid molecules” “oligonucleotide”, “polynucleotide”, and “nucleotides” may interchangeably be used. The terms are directed to polymers of deoxyribonucleotides (DNA), ribonucleotides (RNA), and modified forms thereof in the form of a separate fragment or as a component of a larger construct, linear or branched, single stranded, double stranded, triple stranded, or hybrids thereof. The term also encompasses RNA/DNA hybrids. The polynucleotides may include sense and antisense oligonucleotide or polynucleotide sequences of DNA or RNA. The DNA molecules may be, for example, but not limited to: complementary DNA (cDNA), genomic DNA, synthesized DNA, recombinant DNA, or a hybrid thereof. The RNA molecules may be, for example, but not limited to: ssRNA or dsRNA and the like. The terms further include oligonucleotides composed of naturally occurring bases, sugars, and covalent internucleoside linkages, as well as oligonucleotides having non-naturally occurring portions, which function similarly to respective naturally occurring portions. The terms “nucleic acid segment” and “nucleotide sequence segment,” or more generally “segment,” will be understood by those in the art as a functional term that includes both genomic sequences, ribosomal RNA sequences, transfer RNA sequences, messenger RNA sequences, operon sequences, and smaller engineered nucleotide sequences that are encoded or may be adapted to encode, peptides, polypeptides, or proteins. All nucleic acid primers are presented in the 5′ to 3′ prime direction unless otherwise noted.

The term “gene” or “sequence” refers to a coding region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (down-stream) the coding region (open reading frame, ORF) as well as, where applicable, intervening sequences (i.e., introns) between individual coding regions (i.e., exons). The term “structural gene” as used herein is intended to mean a DNA sequence that is transcribed into mRNA which is then translated into a sequence of amino acids characteristic of a specific polypeptide. It should be noted that any reference to a SEQ ID, or sequence specifically encompasses that sequence, as well as all corresponding sequences that correspond to that first sequence. For example, for any amino acid sequence identified, the specific specifically includes all compatible nucleotide (DNA and RNA) sequences that give rise to that amino acid sequence or protein, and vice versa.

Loop-mediated isothermal amplification (LAMP): A method for amplifying DNA. The method is a single-step amplification reaction utilizing a DNA polymerase with strand displacement activity (e.g., Notomi et al., Nucl. Acids. Res. 28:E63, 2000; Nagamine et al., Mol. Cell. Probes 16:223-229, 2002; Mori et al., J. Biochem. Biophys. Methods 59:145-157, 2004). At least four primers, which are specific for eight regions within a target nucleic acid sequence, are typically used for LAMP. The primers include a forward outer primer (F3), a reverse outer primer (R3), a forward inner primer (FIP), and a backwards inner primer (BIP). A forward loop primer (LF), and a backwards loop primer (LB) can also be included in some embodiments. The amplification reaction produces a stem-loop DNA with inverted repeats of the target nucleic acid sequence. Reverse transcriptase can be added to the reaction for amplification of RNA target sequences. This variation is referred to as RT-LAMP.

The term “infection,” or “infect” as used herein is directed to the presence of a microorganism within a subject body and/or a subject cell. For example, a virus may be infecting a subject cell.

As used herein, the term “asymptomatic” refers to an individual who does not exhibit physical symptoms characteristic of being infected with a given pathogen, or a given combinations of pathogens.

Some embodiments of the invention comprise detecting in a sample from a patient, a level of a biomarker, wherein the presence or expression levels of the biomarker are indicative of infection or possible infection by one or more pathogens. As used herein, the term “biological sample” or “sample” includes a sample from any bodily fluid or tissue. Biological samples or samples appropriate for use according to the methods provided herein include, without limitation, blood, serum, urine, saliva, tissues, cells, and organs, or portions thereof.

A “subject” is any organism of interest, generally a mammalian subject, and preferably a human subject.

The term “expression,” as used herein, or “expression of a coding sequence” (for example, a gene or a transgene) refer to the process by which the coded information of a nucleic acid transcriptional unit (including, e.g., genomic DNA or cDNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s).

Any isothermal amplification protocol can be used according to the methods provided herein. Exemplary types of isothermal amplification include, without limitation, nucleic acid sequence-based amplification (NASBA), loop-mediated isothermal amplification (LAMP), strand displacement amplification (SDA), helicase-dependent amplification (HDA), nicking enzyme amplification reaction (NEAR), signal mediated amplification of RNA technology (SMART), rolling circle amplification (RCA), isothermal multiple displacement amplification (EVIDA), single primer isothermal amplification (SPIA), recombinase polymerase amplification (RPA), and polymerase spiral reaction (PSR, available at nature.com/articles/srepl2723 on the World Wide Web). In some cases, a forward primer is used to introduce a T7 promoter site into the resulting DNA template to enable transcription of amplified RNA products via T7 RNA polymerase. In other cases, a reverse primer is used to add a trigger sequence of a toehold sequence domain.

As used herein, the term “amplified” refers to polynucleotides that are copies of a particular polynucleotide, produced in an amplification reaction. An amplified product, according to the invention, may be DNA or RNA, and it may be double-stranded or single-stranded. An amplified product is also referred to herein as an “amplicon”. As used herein, the term “amplicon” refers to an amplification product from a nucleic acid amplification reaction. The term generally refers to an anticipated, specific amplification product of known size, generated using a given set of amplification primers.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

### Example 1: Summary of Inventive Features

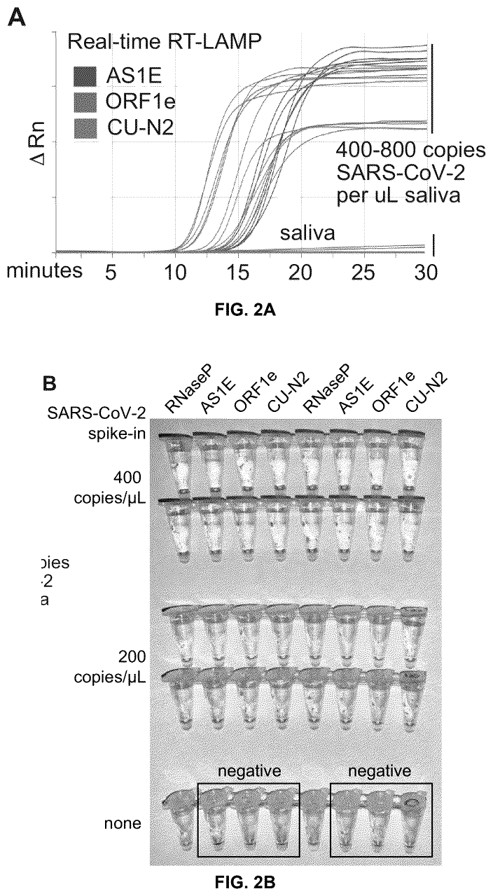

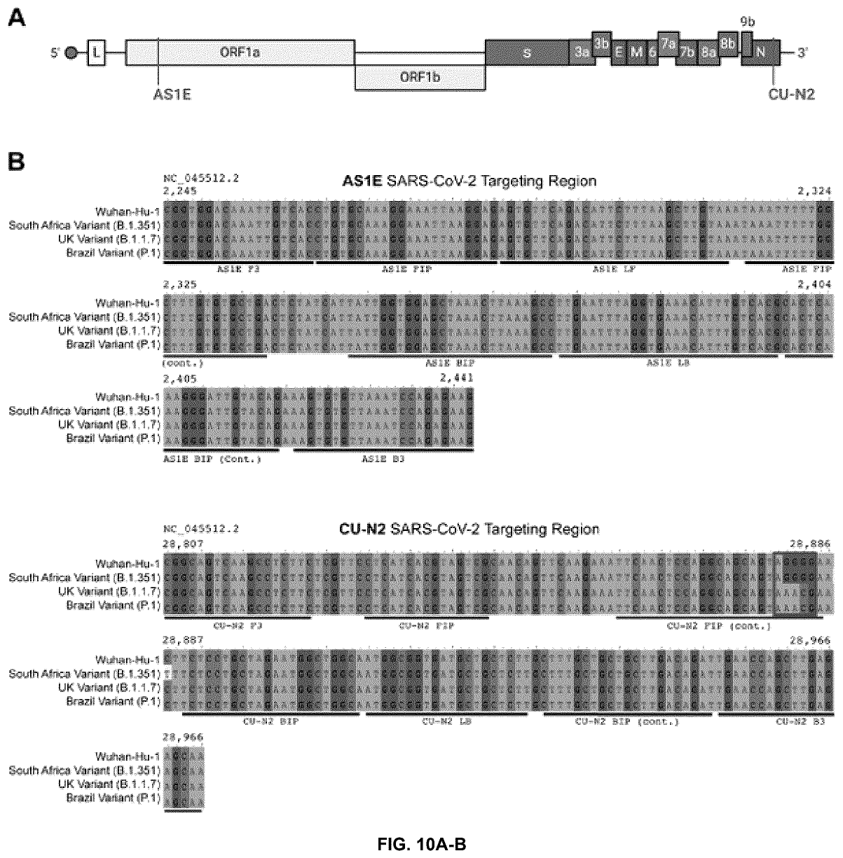

In one embodiment, the results below describe a simple molecular test for SARS-CoV-2 in saliva based on reverse transcription loop-mediated isothermal amplification. The test has two steps. (1) heat saliva with a stabilization solution and (2) detect virus by incubating with a primer/enzyme mix. After incubation, saliva samples containing the SARS-CoV-2 genome turn bright yellow. Because this test is pH dependent, it can react falsely to some naturally acidic saliva samples. The present inventors report unique saliva stabilization protocols that rendered 295 healthy saliva samples compatible with the test, producing zero false positives. We also evaluated the test on 278 saliva samples from individuals who were infected with SARS-CoV-2 but had no symptoms at the time of saliva collection, and from 54 matched pairs of saliva and anterior nasal samples from infected individuals. The Saliva TwoStep test described herein identified infections with 94% sensitivity and >99% specificity in individuals with sub-clinical (asymptomatic or pre-symptomatic) infections. There are several advantages to the SARS-CoV-2 Saliva TwoStep RT-LAMP screening approach described herein: (1) The use of saliva eliminates invasive nasal swab-based sampling, which requires special supplies and causes discomfort. (2) We introduced a saliva stabilization solution that allows for analysis of a broad range of naturally acidic saliva samples while maintaining compatibility with a colorimetric RT-LAMP assay. The solution also helps preserve saliva samples for at least 4 days before processing and lowers saliva viscosity. (3) We demonstrated sample heating condition that liberates the host and viral RNA with minimal degradation. The simple heating step increases biosafety and avoids formal RNA extraction procedures. (4) For RT-LAMP, we incorporated additional primers based on up-do-date SARS-CoV-2 genome databases and identified primers allowing efficient target amplification. These primers are expected to work on most or all viral variants currently circulating (FIGS. 2, and 10). Overall, with the simplified two steps of saliva preparation and virus detection, the test has a rapid sample-to-result turnaround time of 45 min.

The present inventors further identified other potential sources of false-positive results and provided a detailed summary for troubleshooting (Materials and methods and Appendix 1, below and incorporated previously by reference). In addition, from our experience of the actual deployment of this screening test, we summarized the standard operational procedures for saliva sample collection, including the design of a stabilization solution dispensing apparatus to preserve samples while avoiding environmental contamination and protecting workers (Figured 6, and 15; Appendix 1, incorporated herein by reference). By strictly following these application notes, we completely avoided false positive results during the evaluation of a large cohort of human saliva samples, achieving 100% specificity. We also evaluated the RT-LAMP test performance based on the experimentally determined limit of detection. Using SARS-CoV-2-positive human saliva samples, we confirmed that the RT-LAMP test can consistently identify infected individuals with 94% sensitivity.

The present inventors also explored additional methods that may help enhance the RT-LAMP test performance and consistency. Previous work suggests that the addition of 40 mM of guanidine chloride in the RT-LAMP reaction mix could increase RT-LAMP amplification efficiency. However, we did not observe similar enhancement when included in our experiments. To further prevent carry over contamination, the usage of dUTP and uracil-DNA-glycosylase-containing RT-LAMP reaction mix can be considered. Previous studies have shown that the addition of this alternative master mix does not affect the RT-LAMP limit of detection.

Saliva TwoStep requires less sample processing, reaction incubation time, and laboratory overhead as compared to quantitative RT-PCR methods. The result is the ability to run significantly more tests with a given amount of resources. Based on these observations, we conclude that the Saliva TwoStep test described herein can be used as a SARS-CoV-2 screening tool to reliably identify infectious individuals with minimal laboratory setup, potentially serving as a tool for effective SARS-CoV-2 surveillance at the community level. This RT-LAMP testing offers many solutions to a nation-wide shortage of COVID-19 testing. With minimal set-up, this test could be performed in diverse settings such as factories, office buildings, or schools.

### Example 2: Optimized Universal Saliva Stabilization Conditions for RT-LAMP

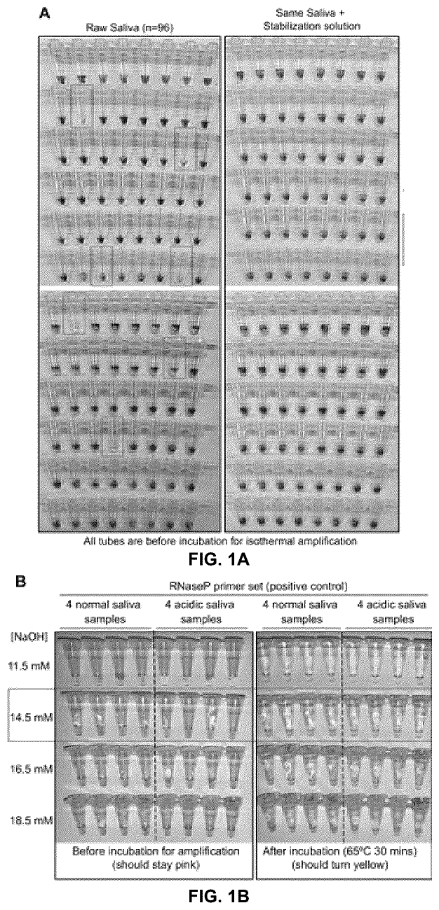

To deal with the variability in pH of human saliva, the present inventors (also referred to herein as “we”) optimized a basic saliva stabilization solution by titrating in various concentrations of sodium hydroxide (NaOH). We performed this optimization using a control RT-LAMP primer set, ‘RNaseP’, which amplifies the mRNA transcript produced from the human POP7 gene (primer set developed previously. One goal of this embodiment was to increase the pH of all saliva samples well above the indicator flip-point of pH 6.8, while not making the samples so basic that they could not reach this pH upon successful target amplification. We found that human saliva containing 14.5 mM NaOH is preferred to inhibit false positives caused by saliva acidity (N=96; FIG. 1A, right) without impeding the intended color change during amplification (FIG. 1B). In addition, we designed our saliva stabilization solution to also include a chelating agent (1 mM ethylenediaminetetraacetic acid [EDTA] final concentration) and Proteinase K to inhibit RNases, both of which help preserve virion RNA and therefore to increase sensitivity. (Note that Proteinase K will inhibit the RT-LAMP reaction if it does not go through a heat inactivation step prior to that reaction.) Finally, the saliva stabilization solution contains TCEP, which aids in RNA stabilization by breaking disulfide bonds present in RNases and helps to reduce saliva viscosity. Our optimized saliva stabilization solution (2× solution: 5 mM TCEP, 2 mM EDTA, 29 mM NaOH, 100 μg/mL Proteinase K, diluted in DEPC-treated water, also referred to as nuclease-free water) is provided in one embodiment of the invention.

### Example 3: Optimized RT-LAMP Primer Sets for Detecting SARS-CoV-2 in Human Saliva

A critical parameter in RT-LAMP is primer design because RT-LAMP requires four to six primers all working together. We found that the ‘AS1E’ set, developed by Rabe et al. and targeting the ORF1ab region of the SARS-CoV-2 genome, performs very well. However, in order to target two distinct regions from the SARS-CoV-2 genome, we designed and tested a large number of additional primer sets. Two of our custom sets, ‘ORF1e’ targeting the virus ORF1ab gene and ‘CU-N2’ targeting the virus N gene, exhibited similar sensitivity and amplification efficiency as the AS1E set, as determined using real-time fluorescence monitoring of RT-LAMP products (FIGS. 2A, and 10). We next confirmed that these primer sets were both compatible with saliva preserved in our saliva stabilization solution and with colorimetric RT-LAMP (FIG. 2B).

### Example 4: Sample Heat Inactivation

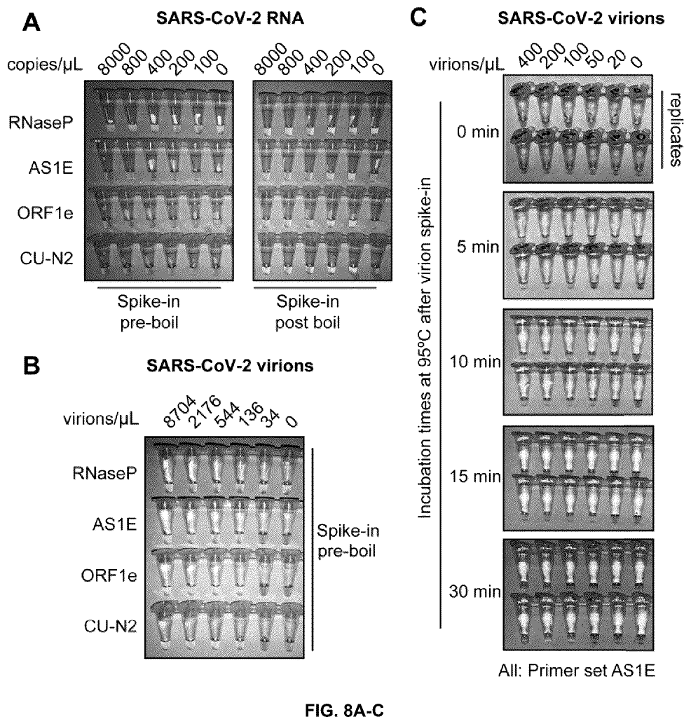

Next, we addressed the biosafety concerns of handling potentially infectious saliva samples. Recent studies suggest that incubation for 3 min at 95° C. is sufficient to inactivate SARS-CoV-2 virions. However, when heating saliva samples for downstream analysis of RNA, one must balance heating long enough to liberate the target RNA from virions with not heating for so long that the target RNA will be degraded. Heating at 95° C. does degrade SARS-2-COV RNA that is spiked directly into saliva samples but does not degrade viral RNA when it is spiked into samples within SARS-CoV-2 virions (FIG. 8). A 10 min incubation of saliva samples at 95° C. was found to be preferred in one embodiment (FIG. 8). We designed our test procedure such that testing personnel avoid handling open tubes until after this step to increase biosafety (Appendix 1, previously incorporated by reference).

### Example 5: Assessment of Sample Stability During Storage

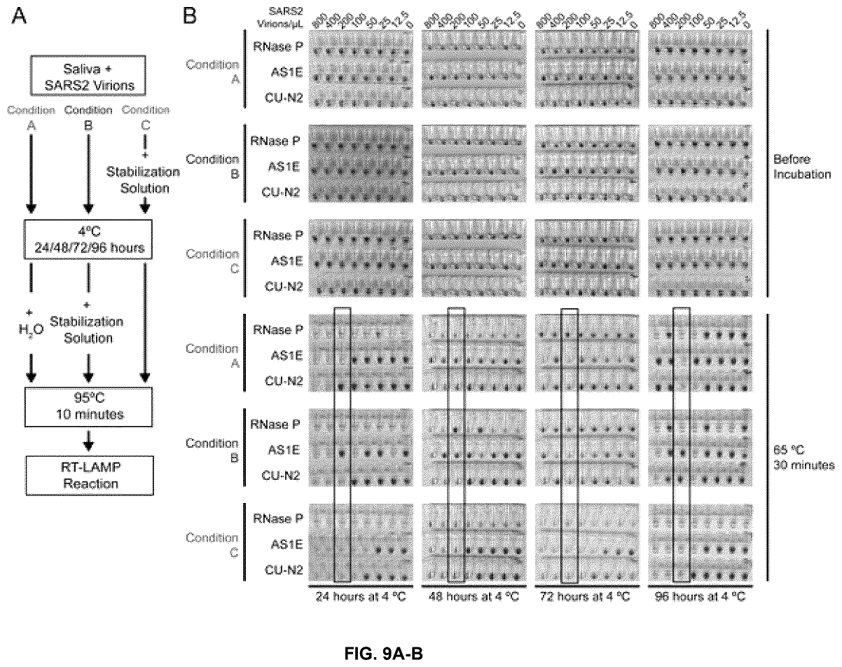

Stability of saliva samples from the time of collection to the time of processing and analysis is important if testing cannot be performed immediately or if the tests are being conducted in batches. Saliva samples containing purified virions and diluted with 2/saliva stabilization solution were stored at 4° C. for 24, 48, 72, or 96 hr before being inactivated at 95° C. and analyzed using colorimetric RT-LAMP (FIG. 9) We tested saliva collection and storage over a range of SARS-CoV-2 virion spike-in concentrations. We observed no significant changes in sample stability and the test detection limit over this time course, suggesting that saliva samples stored in saliva stabilization solution at 4° C. are stable for at least 4 days.

### Example 6: Determining the Limit of Detection

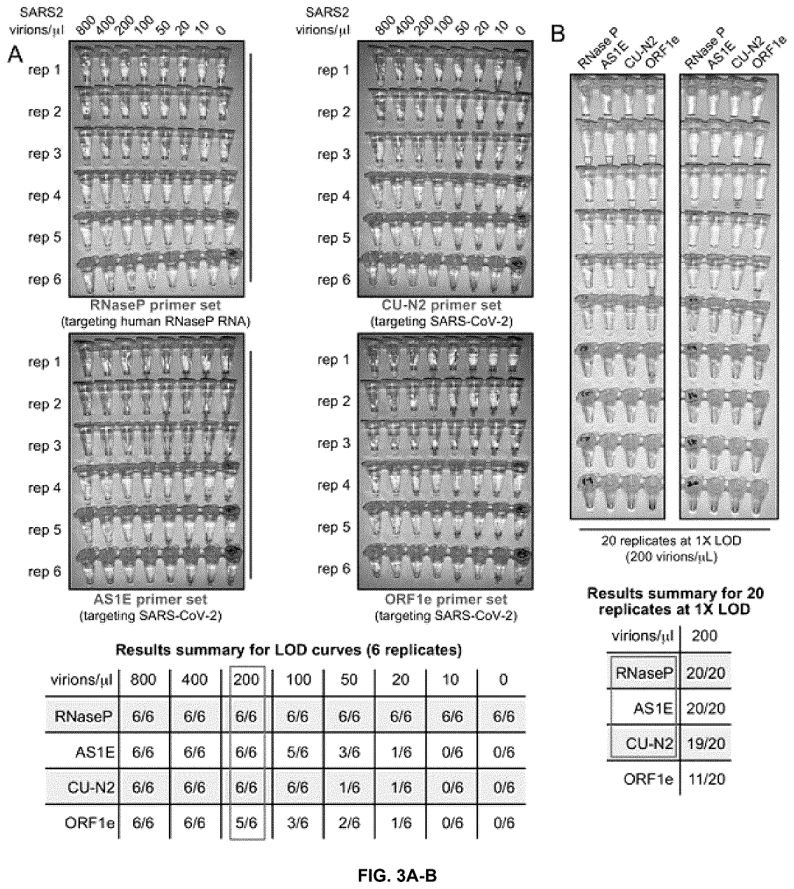

We next determined the limit of detection for this test. The lowest concentration at which positive samples were reliably identified was 200 virions/μL in saliva (red box, summary table in FIG. 3A) Note that the limit of detection refers to the virus concentration that can be identified >95% of the time, and the assay does often detect the virus at even lower concentrations. We next tested 20 replicates at this concentration (200 virions/μL) using all four primer sets (FIG. 3B). The ORF1e primer set was not consistent in its performance at 200 virions/μL. Therefore, we decided to eliminate the ORF1e primer set from our testing panel from this point forward.

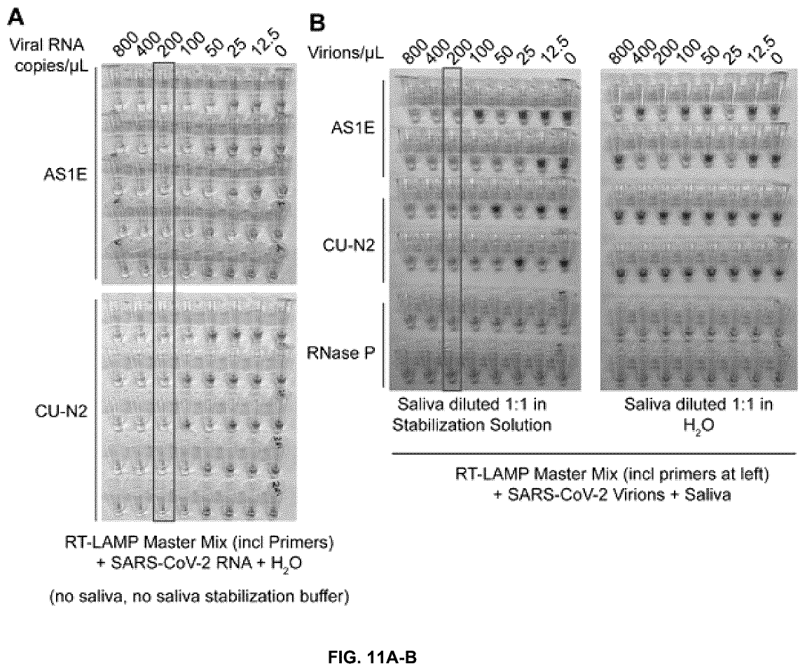

We considered that contaminants in saliva and/or components of the saliva stabilization solution might be suppressing the overall RT-LAMP reaction efficiency by acting in inhibitory ways. On the contrary, we found that when synthetic SARS-CoV-2 RNA is directly added to the RT-LAMP reaction mix (in the absence of saliva and the stabilization solution), we were unable to achieve a better detection limit than 200 genome copies/μL (FIG. 11). This suggests that the observed detection limit represents the upper performance limit of the presented RT-LAMP assay, and that the saliva and stabilization solution have little to no negative impact to the test performance. In fact, multiple observations suggest that RNA degradation is observed in the absence of stabilization solution, resulting in less consistent testing results (FIG. 9; FIG. 11)

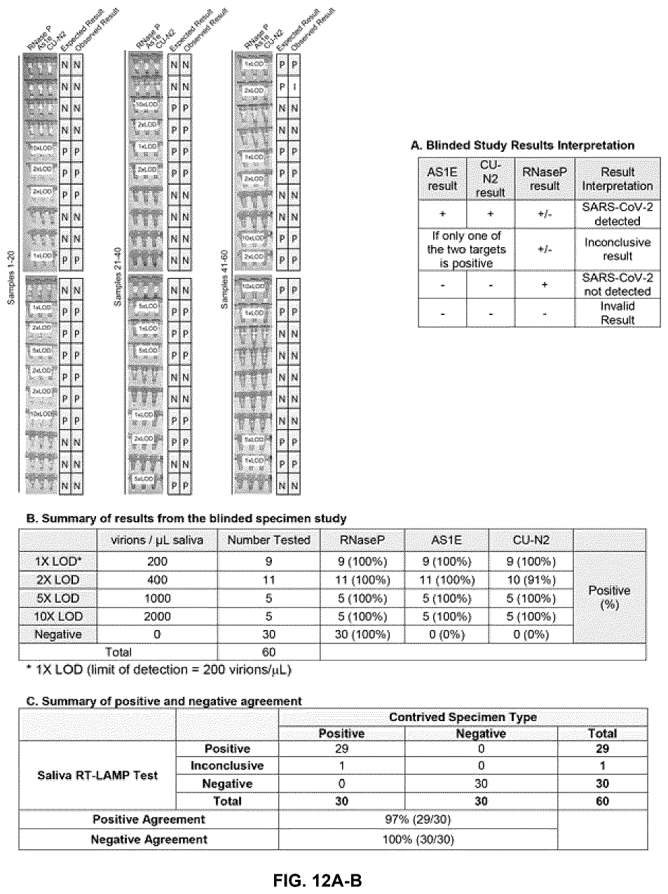

We next performed a blinded study. Heat-inactivated virions were spiked into human saliva at various concentrations at or above the limit of detection (200 virions/μL), and these as well as uninfected saliva samples were blinded and passed to a second member of our personnel. After running the RT-LAMP test on 60 such samples, only one positive sample scored as inconclusive. In that sample the SARS-CoV-2 primer set CU-N2 failed, while the other primer set detecting SARS-CoV-2 correctly identified the sample (FIG. 12). All negative samples were scored correctly (100% specificity, binomial 95% confidence interval [88%, 100%]). Conservatively counting the inconclusive test as a false negative lead to a sensitivity estimate of 97% (binomial 95% confidence interval [93%, 100%]). See FIG. 4C for a breakdown by primer set.

### Example 7: Evaluation on Human Samples

SARS-CoV-2 screening was initiated on the University of Colorado Boulder campus starting in the summer/fall of 2020. Saliva samples were taken weekly from residents of dormitories and at several testing sites throughout the campus. Participants were asked to refrain from eating or drinking 30 min prior to sample collection, and to not participate if they were experiencing any symptoms consistent with COVID-19. Therefore, individuals testing positive for SARS-CoV-2 were either pre-symptomatic at the time of saliva collection, or they never developed symptoms throughout the course of infection (we do not have the necessary follow-up data to delineate). All saliva samples were analyzed by a quantitative RT-PCR method performed directly on saliva, which was mixed 1:1 with 2×TBE buffer containing 1% Tween-20 (saliva sample prep as in Ranoa et al., 2020, such procedures being incorporated herein by reference). An in-house multiplex quantitative RT-PCR assay was used to determine the presence of the SARS-CoV-2 genome in saliva, with primers targeting the E and N gene regions of the SARS-2-COV genome (see Materials and methods). Because positive results in our university screening regimen resulted in the tested individual being directed to their healthcare provider for confirmatory testing, positively tested individuals were removed from the sampling pool. Thus, most positive samples are from unique individuals, with a few exceptions.

All positive saliva samples with viral load above the quantitative RT-PCR detection limit (collected between Sep. 16-25, 2020; N=278), along with 295 negative saliva samples, were next re-evaluated with RT-LAMP. Saliva samples had already been heat inactivated for 30 min at 95° C. as the initial step of the quantitative RT-PCR protocol. Since the heating component of our Saliva Preparation step had already been performed, an aliquot of the heated saliva sample was transferred into our 2× saliva stabilization solution (without Proteinase K) and then put through the RT-LAMP reaction as described above. For each of the 573 samples, three RT-LAMP reactions were performed with different primer sets: RNaseP (positive control), AS1E, and CU-N2 (the latter two sets detecting SARS-CoV-2). During this part of the study, we noticed that decreasing the input sample amount (saliva+saliva stabilization solution) from 4 μL to 2 μL in a total reaction volume of 20 μL further increased tolerance of the RT-LAMP reaction color to acidic saliva samples because less saliva is added. We thus reduced the input sample amount to 2 μL when evaluating these human samples. For all 573 samples, RT-LAMP with primers recognizing a human RNA positive control (RNaseP) correctly turned positive (yellow).

### Example 8: Specificity and Sensitivity

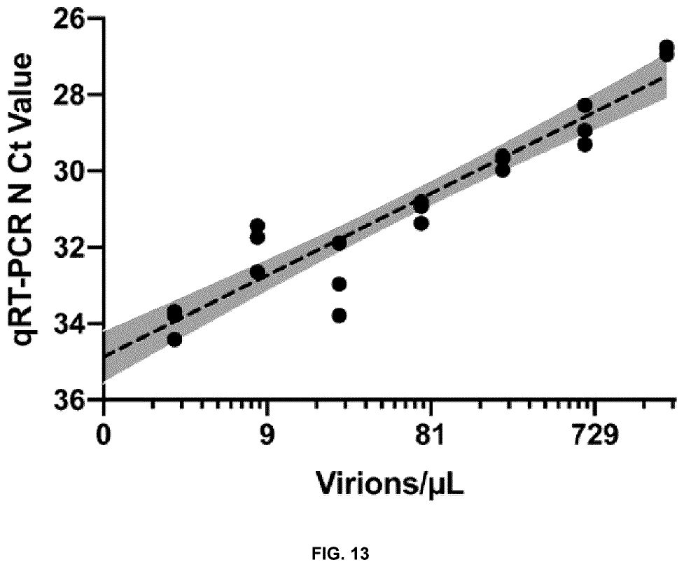

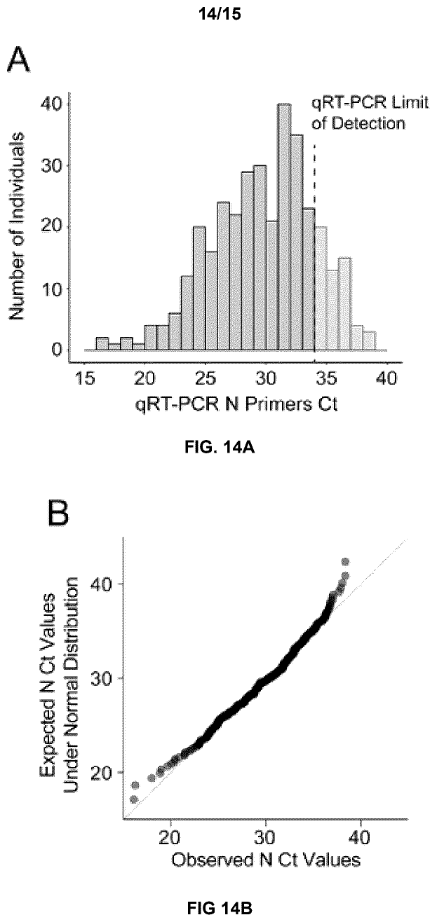

Two hundred and ninety-five saliva samples that tested negative for SARS-CoV-2 by quantitative RT-PCR were used for evaluation. We re-tested all of those samples with RT-LAMP to evaluate our false-positive rate. For all 295 SARS-CoV-2-negative samples, the AS1E and CU-N2 primer sets both returned a result of negative, consistent with the quantitative RT-PCR results. Therefore, there were zero false positives, and the test had a specificity of 100% in this extensive sample set. This shows the strength of our saliva stabilization solution, which mitigates the problem of false positives in RT-LAMP due to some human saliva samples being naturally acidic. We next re-analyzed 278 SARS-CoV-2-positive saliva samples using RT-LAMP. We determined the viral load of each positive saliva sample using the quantitative RT-PCR standard curve generated by our university testing lab (FIG. 13). All viral load and Ct values reported in this study are from the nucleocapsid (N) primer set (FIG. 14). Among all positive samples, 208 (74.8%; AS1E primers) and 182 (65.5%; CU-N2 primers) returned positive RT-LAMP test results (FIG. 4A). Although both primer sets were still able to detect SARS-CoV-2 RNA below the experimentally determined detection limit (200 virions/μL), we observed a decline in the test sensitivity below such limit (FIG. 4B). The observed limit of detection of the AS1E primer set was determined from this data to be 266 virions/μL. The strong congruence with our prior estimate of 200 virions/AL demonstrates that heating for 30 min prior to adding stabilization solution and using 2 μL of saliva plus stabilization solution, instead of 4 μL, both have very little effect. Of the 168 positive samples with viral loads greater than RT-LAMP limit of detection, 158 (94%; AS1E primers) or 142 (85%; CU-N2 primers) returned positive RT-LAMP test results (FIG. 4A). In FIG. 4C, we summarize the performance of each primer set in both this test of human saliva samples, and in the blinded virion spike-in experiment described above (FIG. 12). Because the AS1E primer set performs best throughout our study, we include that as the main primer set in our final test configuration, which we refer to as the Saliva TwoStep test. However, the CU-N2 primer set still performs well and can be used when it is desirable to detect a second region of the SARS-CoV-2 genome.

### Example 9: Test Sensitivity as a Function of Viral Load in the Sample

For both primer sets, we calculated the sensitivity (positive agreement with quantitative RT-PCR) and specificity (negative agreement with quantitative RT-PCR) of the RT-LAMP test at various levels of viral load cutoffs (FIG. 4B, Table 1). The differences in the observed limit of detection between the two SARS-CoV-2 primer sets could reflect the differences in the primer efficiencies, as well as the dynamics in relative viral transcript abundance.

### Example 10: Assessment of Saliva Two-Step Against a Nasal Swab Test

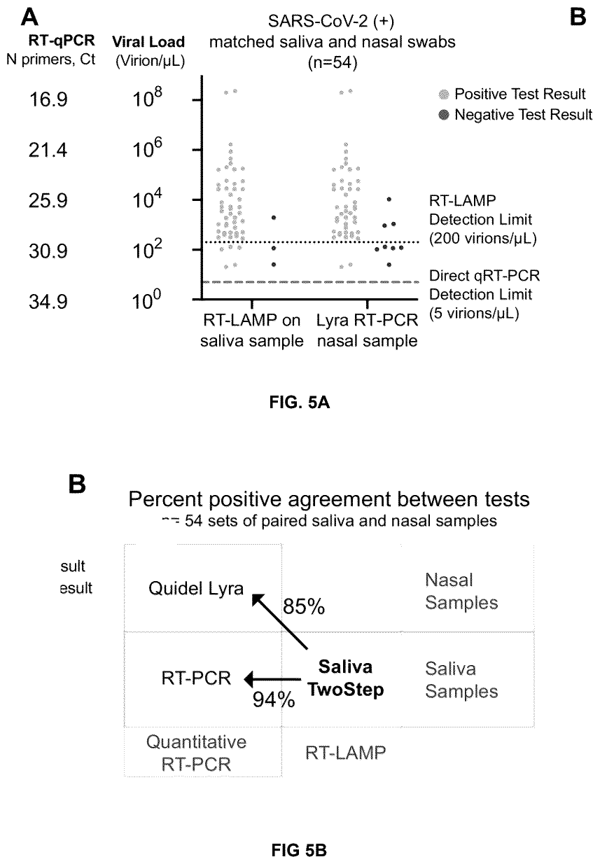

Of the 278 SARS-CoV-2-positive saliva samples analyzed above, 54 also had matched nasal samples collected no more than 2 days later. In some cases, individuals may have developed symptoms by the time follow-up nasal swabs were taken, so we can make no claims about symptomatic status at the time of nasal swab. We next compared the results of the Saliva TwoStep test with the results obtained by the Quidel Lyra direct nasal swab RT-PCR test. Compared to the quantitative RT-PCR on saliva results, the RT-LAMP produced three false negatives in this sample set, whereas the Lyra nasal swab test produced eight (Figure SA). However, this is still remarkably consistent given that this comparison involves three degrees of freedom: biosample (saliva versus nasal swab), test modality (RT-PCR versus RT-LAMP), and days between saliva and nasal samples collection (up to 2 days apart). A summary of how these first two degrees of freedom affect test congruency are shown in FIG. 5B.

### Example 11: Optimized Test Conditions

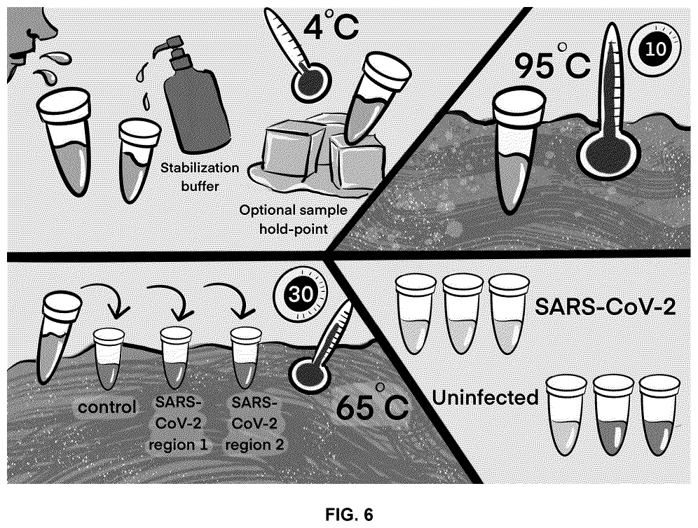

From the experiments described above, we selected the final optimized conditions for our Saliva TwoStep test. The two steps have an end-to-end processing and analysis time of approximately 45 min (FIG. 6). For additional application details regarding the testing station setup, sample collection, and overall workflow of employing this test for community screening, please refer to Appendix 1, electronically available at the following https://elifesciences.org/articles/65113#content, being incorporated herein by reference).

In one embodiment, an optimized sample test may include one or more of the following steps: (Step 1) Prepare saliva: Collect saliva combine 1:1 with 2/saliva stabilization solution and incubate at 95° C. for 10 min. (We have determined that performing a heating step at 95° C. for 30 min in a water bath, before addition of the saliva stabilization solution, also works reasonably well.) However, in this case, Proteinase K must be omitted. (Step 2) Detect virus. Incubate at 65° C. for 30 min: 2 μL diluted saliva from step 1, 10 μL 2×NEB Colorimetric RT-LAMP enzyme mix, 6 μL of nuclease-free water, and 2 μL. 10× primer mix for a final reaction volume of 20 μL. (Step 3) Reaction inactivation (optional) Stop reaction at 80° C. for 2 min. This stabilizes color so that results can be analyzed at a later time. The multiple heating steps here may be programmed into a thermal cycler for maximum convenience, but this is not necessary.

### Example 12: Materials and Methods

RT-LAMP primer design and preparation: Regions of the SARS-CoV-2 genome that are conserved among strains were identified using genome diversity data from NextStrain (nextstrain.org/ncov/global). Next, nucleotide-BLAST (blast.ncbi.nlm.nih.gov) was used to filter out genome sequences that share high-sequence homology with other seasonal coronavirus genomes. Finally, PrimerExplorer V5 (primerexplorer.jp/e/) was used to design RT-LAMP primers targeting the specific regions of SARS-CoV-2 genomes. The F3, B3, FIP, BIP, Loop F, and Loop B primers were selected for optimal melting temperature and complementarity using A plasmid editor (ApE). In all cases, a 10× concentration of primer sets was made containing 16 μM FIP and BIP primers, 4 μM LF and LB primers, and 2 μM F3 and B3 primers.

All primers (see Table 2 below) should be ordered with HPLC purification, which ensures the yield and avoids cross-contamination from other SARS-CoV-2-related synthesis projects being run on the same equipment at the oligo synthesis facilities (which can lead to false positives). This is particularly a problem during a pandemic where these facilities are handling many oligo synthesis orders focused on the same pathogen (It is also recommended that you communicate with the primer synthesis company to inform them that primers are intended for use with a SARS-CoV-2 screening test. Several companies have dedicated facilities for minimizing cross-contamination of SARS-CoV-2 templates. In addition, primers should be diluted in nuclease-free water, instead of Tris-EDTA buffer, which will also inhibit pH change that takes place during RT-LAMP.

SARS-CoV-2 RNA and virion standards: Synthetic SARS-CoV-2 RNA control (Twist Bioscience #102019) was obtained, and its copy number of 1×10⁶ copies/μL was confirmed using quantitative RT-PCR in conjunction with a DNA plasmid control containing a region of the N gene from the SARS-CoV-2 genome (IDT #10006625). Heat-inactivated SARS-CoV-2 virion control (ATCC #VR-1986HK) was obtained and its concentration of 3.75×10⁵ virions/μL was confirmed using quantitative RT-PCR in conjunction with both the synthetic SARS-CoV-2 RNA control and a DNA plasmid control containing a region of the N gene from the SARS-CoV-2 genome. SARS-CoV-2 RNA was added to saliva samples after they had been mixed 1:1 with saliva stabilization solution and heated at 95° C. for 10 min, unless stated otherwise, whereas heat-inactivated SARS-CoV-2 virions were added directly to saliva samples and then mixed 1:1 with saliva stabilization solution before being heated. Concentrations reported throughout this study represent the final concentration of standards in saliva before it was mixed 1:1 with 2× saliva stabilization solution.

Saliva Preparation with Heat and Stabilization Solution:

In one embodiment a 2× saliva stabilization solution (see Table 3) may include: (1) TCEP-HCl (GoldBio #TCEP10). The -HCl form can be used to produce the correct final stock pH. (2) EDTA, 0.5 M, pH 8.0 (Sigma-Aldrich #324506). It is advantageous in this embodiment to use a pH 8.0 stock solution, otherwise this also affects the pH of the final stabilization solution. (3) lyophilized Proteinase K (Roche #3115879001). It is advantageous to use the lyophilized form. Liquid forms will contain Tris, which inhibits the pH change during the RT-LAMP reaction. (4) Ten molar NaOH was prepared by dissolving NaOH pellets (Sigma-Aldrich #221465) into nuclease-free water, before being added to the 2× solution to reach the correct concentration.

Saliva samples (1 mL) were collected in sterile, nuclease-free 5 ml conical screw-cap tubes (TLD Five-O) #TLDC2540). 2× saliva stabilization solution described above was then added at a 1:1 ratio. Samples were shaken vigorously for 5-10 s and incubated at 95° C. for 10 min. Samples were then placed on ice before being used in downstream analyses (Detailed sample collection procedure is described in Appendix 1, previously incorporated by reference.)

Real-time RT-LAMP: For each reaction, 10 μL WarmStart LAMP 2/Master Mix (NEB #E1700) was combined with 1 μL 20× EvaGreen Dye (Biotium #31000), 2 μl. 10× primer mix, and 3 μL DEPC-treated water. The combined reaction mix was added to MicroAmp Optical 96-Well Reaction Plate (ThermoFisher #N8010560), and then 4 μL processed saliva sample was added. The reaction was mixed using a multi-channel pipette and incubated in Applied Biosystems QuantStudio3 Real-time PCR system. The reaction proceeded at 65° C. for 30 min with fluorescent signal being captured every 30 s. The results were visualized and analyzed using ThermoFisher's Design and Analysis software.

Colorimetric RT-LAMP: WarmStart Colorimetric LAMP 2× Master Mix (NEB #M1800) was used in all colorimetric RT-LAMP reactions. Each reaction was carried out in a total of 20 μL (10 μL WarmStart Master Mix, 2 μL 10× primer mix, 4 μL processed saliva sample, and 4 μL DEPC-treated water). Reactions were set up in PCR strip tubes on ice. Saliva template was added last, and tubes were inverted several times to mix samples and briefly spun down in a microfuge. Reactions were incubated in a thermal cycler at 65° C. for 30 min and then deactivated at 80° C. for 2 min. The incubation was carried out without the heated lid to simulate a less complex heating device. Images of reactions were taken using a smartphone. For the community deployment of this assay, 2 μL of processed saliva was used instead of 4 μL.

Testing of university samples: The University of Colorado Boulder SARS-CoV-2 screening test was loosely based on a published quantitative RT-PCR performed directly on saliva (Ranoa et al., 2020), which has a limit of detection of 5 virions/μL. Some modifications were made, as described here. For sample collection, individuals were asked to collect no less than 0.5 mL of saliva in a 5 mL screw-top collection tube. Saliva samples were heated at 95° C. for 30 min to inactivate the viral particles for safe handling and then placed on ice or at 4° C. For quantitative RT-PCR analysis, the university testing team transferred 75 μL of saliva into a 96-well plate, where each well had been pre-loaded with 75 μL 2×TBE buffer supplemented with 1% Tween 20. (The remaining saliva in the 5 mL collection tube proceeded to RT-LAMP testing as described in the next paragraph.) Next, 5 μL of this diluted sample was added to a separate 96-well plate where each well had been pre-loaded with 15 μL reaction mix composed of TaqPath 1-step Multiplex Master Mix (Thermo Fisher A28523), nuclease-free water, and triplex primer mix consisting of primer and probe sets targeting SARS-CoV-2 E and N genes and human RNase P gene (sequence and concentration specified in the table below). The reactions were mixed, spun down, and loaded onto a Bio-Rad CFX96 or CFX384 qPCR machine. Quantitative RT-PCR was run using the standard mode, consisting of a hold stage (25° C. for 2 min, 50° C. for 15 min, and 95° C. for 2 min) followed by 44 cycles of a PCR stage (95° C. for 3 s, 55° C. for 30 s, with a 1.6° C./s ramp up and ramp down rate) Only Ct values from the N primer set are reported in the study herein and used to calculate relative sample viral load based on the standard curve shown in FIG. 13.

Leftover samples from this testing procedure were then tested with RT-LAMP. Fifty microliters of saliva was transferred and mixed into a 96-well plate, where each well had been pre-loaded with 50 μL 2× saliva stabilization solution without proteinase K (5 mM TCEP-HCl, 2 mM EDTA, 29 mM NaOH, diluted in DEPC-treated water). Two microliters of this diluted saliva was transferred into eight-strip PCR tubes containing RT-LAMP reaction mixture (enzymes and primers provided at elifesciences.org/articles/65113 #content, and specifically incorporated herein by reference). For each sample, three RT-LAMP reactions were carried out to amplify human RNaseP as a control and AS1E and CU-N2 for SARS-CoV-2. The reactions were incubated at 65° C. for 30 min followed by inactivation at 80° C. for 2 min on a thermal cycler (Bio-RAD T100). A color change from pink to yellow was observed visually to interpret results.

### TABLES

TABLE 1
Summary of RT-LAMP evaluation in human samples.

| | | | RT-LAMP Primers | | | | |
|---|---|---|---|---|---|---|---|
| | | | A51E | | | CU-N2 | |
| | | | No. of | No. of | | No. of | |
| | | | samples | positives | Agreement | positives | Agreement |
| Quantitative | Negative | | 295 | 0 | 295/295 (100%) | 0 | 295/295 (100%) |
| RT-PCR | Positive | 4000 | 82 | 82 | 82/82 (100%) | 82 | 82/82 (100%) |
| (SARS-CoV-2 N) | (levels of viral load: | 2000 | 97 | 97 | 97/97 (100%) | 94 | 94/97 (96.9%) |
| | Virions/μL) | 1000 | 118 | 117 | 117/118 (99.2%) | 110 | 110/118 (93.2%) |
| | | 800 | 123 | 122 | 122/123 (99.2%) | 112 | 112/123 (91.1%) |
| | | 400 | 143 | 139 | 139/143 (97.2%) | 129 | 145/173 (90.2%) |
| | | 200 | 168 | 158 | 158/168 (94.0%) | 142 | 142/168 (84.5%) |

TABLE 2
RT-LAMP Primers

| Primer | LAMP | | |
|---|---|---|---|
| set | primer | | |
| name | component | Primer Sequence (5′-3′) | |
| ‘RNaseP’ amplifies | F3 | TTGATGAGCTGGAGCCA | SEQ ID NO. 4 |
| human RNA | B3 | CACCCCTCAATGCAGAGTC | SEQ ID NO. 5 |
| for positive control | Loop F | ATGTGGATGGCTGAGTTGTT | SEQ ID NO. 6 |
| (Curtis et al., 2018) | Loop B | CATGCTGAGTACTGGACCTC | SEQ ID NO. 7 |
| | FIP | GTGTGACCCTGAAGACTCGGT | SEQ ID NO. 8 |
| | | TTTAGCCACTGACTCGGATC | |
| | BIP | CCTCCGTGATATGGCTCTTCGT | SEQ ID NO. 9 |
| | | TTTTTTCTTACATGGCTCTGGTC | |
| ‘AS1E’ | F3 | CGGTGGACAAATTGTCAC | SEQ ID NO. |
| (Rabe and Cepko, 2020) | B3 | CTTCTCTGGATTTAACACACTT | SEQ ID NO. |
| | Loop F | TTACAAGCTTAAAGAATGTCTGAACACT | SEQ ID NO. |
| | Loop B | TTGAATTTAGGTGAAACATTTGTCACG | SEQ ID NO. |
| | FIP | TCAGCACACAAAGCCAAAAATTTAT | SEQ ID NO. |
| | | TTTTCTGTGCAAAGGAAATTAAGGAG | |
| | BIP | TATTGGTGGAGCTAAACTTAAAG | SEQ ID NO. |
| | | CCTTTTCTGTACAATCCCTTTGAGTG | |
| ‘CU-N2’ | F3 | CGGCAGTCAAGCCTCTTC | SEQ ID NO. |
| developed herein | B3 | TTGCTCTCAAGCTGGTTCAA | SEQ ID NO. |
| | Loop F | This set does not require | SEQ ID NO. |
| | | a Loop F primer | |
| | Loop B | ATGGCGGTGATGCTGCTCTT | SEQ ID NO. |
| | FIP | TCCCCTACTGCCTGGAGCGT | SEQ ID NO. |
| | | TCCTCATCACGTAGTCG | |
| | BIP | TCTCCTGCTAGAATGGCTGGC | SEQ ID NO. |
| | | ATCTGTCAAGCAGCAGCAAAG | |
| ‘ORF1 [symbol] ’ developed | F3 | GGCTAACTAACATCTTTGGC | SEQ ID NO. |
| herein | B3 | GTCAGCACACAAAGCCCAA | SEQ ID NO. |
| | Loop F | TCTTCAAGCCAATCAAGGAC | SEQ ID NO. |
| | Loop B | TTGTCGGTGGACAAATTGT | SEQ ID NO. |
| | FIP | TCTCTAAGAAACTCTACACCTTCCTT | SEQ ID NO. |
| | | TTTACTGTTTATGAAAAACTCAAACC | |
| | BIP | TATCTCAACCTGTGCTTGTGAAA | SEQ ID NO. |
| | | TTTTAGAATGTCTGAACACTCTCCT | |

[symbol]

indicates data missing or illegible when filed

TABLE 3
Saliva stabilization solution in one embodiment

| | Amount | | Final concentration | |
|---|---|---|---|---|
| Components | mixed | | in 2× stock solution | |
| TCEP-HCl (GoldBio #TCEP10) | 143.3 | mg | 5 | mM |
| 0.5M EDTA, pH 8.0 | 400 | μL | 2 | mM |
| (Sigma-Aldrich #324506) | | | | |
| 10M NaOH (Sigma-Aldrich | 290 | μL | 29 | mM |
| #221465) | | | | |
| Proteinase K (Roche #3115879001) | 10 | mg | 100 | μg/mL |

| | |
|---|---|
| Nuclease-free water | To 100 mL |

TABLE 4
Testing primer protocol.

| Taqman primer/ | | | |
|---|---|---|---|
| probe set | | 1× | |
| target | Primer or probe name | Concentration | Sequence (5′-3′) |
| SARS-CoV-2 | E_Sarbeco_F1 (IDT 10006888) | 400 nM | ACAGGTACGTTAATAGTTAATAGCGT |
| E gene | E_Sarbeco_R2 (IDT 10006890) | 400 nM | ATATTGCAGCAGTACGCACACA |
| | E_Sarbeco_P (IDT Custom) | 200 nM | TexRd. |
| | | | ACACTAGCCATCCTTACTGCGCTTC |
| | | | G-IAbRQSp |
| SARS-CoV-2 | nCOV_N1_F (IDT 10006830) | 500 nM | GACCCCAAAATCAGCGAAAT |
| N gene | nCOV_N1_R (IDT 10006831) | 500 nM | TCTGGTTACTGCCAGTTGAATCTG |
| | nCOV_N1_P (IDT Custom) | 250 nM | HEX-ACCCCGCAT-ZEN-TACGTT |
| | | | TGGTGGACC-IAbkFQ |
| Human | RNaseP_F (IDT 10006836) | 50 nM | AGATTTGGACCTGCGAGCG |
| RNase P | RNaseP_R (IDT 10006837) | 50 nM | GAGCGGCTGCTCCACAA GT |
| | RNase_P_P (IDT 10006838) | 50 nM | HEX-TTCTGACCT-ZEN-GAAGGCT |
| | | | CTGCGCG-IAbkFQ |

TABLE 5: shows a list of control material(s) to be used with an exemplary SARS-CoV-2 RT-LAMP test of the invention and expected positive, negative and failure outputs in one embodiment thereof.

| | External | | 2019 | | Expected |
|---|---|---|---|---|---|
| Control | Control | Used to | nCoV_{—} | | Color |
| Type | Name | Monitor | ORF1ab | RNaseP | Change |
| Positive | nCoVPC | Substantial | + | − | Red to |
| | | reagent | | | Yellow |
| | | failure | | | |
| | | including | | | |
| | | primer | | | |
| | | integrity | | | |
| Negative | NTC | Reagent | − | − | Stays Red |
| | | and/or | | | |
| | | environmental | | | |
| | | contamination | | | |
| Extraction | HSC | Failure in | − | + | Red to |
| | | lysis and | | | Yellow |
| | | extraction | | | |
| | | procedure, | | | |
| | | potential | | | |
| | | contamination | | | |
| | | during | | | |
| | | extraction | | | |

TABLE 6: Shows a summary of the expected interpretation of the SARS-CoV-2 RT-LAMP test of the invention and a recommended course of action based on the test result in one embodiment thereof

| 2019 | | Result | | |
|---|---|---|---|---|
| nCoV_{—} | | Interpre- | | |
| ORF1ab | RNaseP | tation | Report | Actions |
| + | +/− | 2019-nCoV | Positive | Report results to |
| | | detected | 2019-nCoV | CDC and sender |
| − | + | 2019-nCoV | Not | Report results to |
| | | not | detected | sender. Consider |
| | | detected | | testing for other |
| | | | | respiratory viruses. |
| − | − | Invalid | Invalid | Repeat extraction |
| | | Result | | and RT-LAMP. If |
| | | | | the repeated result |
| | | | | remains invalid, |
| | | | | consider collecting |
| | | | | a new specimen from |
| | | | | patient. |

TABLE 7: shows a summary of the expected interpretation of the SARS-CoV-2 M-RT-LAMP test for CoV incorporating the identification of one or more host RNA markers of infection.

| Detection Primer | Reaction | Interpretation |
|---|---|---|
| SARS2-PrimerSet1 | + | Positive infection |
| SARS2-PrimerSet2 | + | Positive infection |
| HostBiomarker | + | Early stage COVID-19 positive |
| HostControl | + | Positive control |
| SARS2-PrimerSet1 | + | Positive infection |
| SARS2-PrimerSet2 | + | Positive infection |
| HostBiomarker | − | Late State / Recovering COVID-19 |
| | | positive |
| HostControl | + | Positive control |
| SARS2-PrimerSet1 | − | Negative infection |
| SARS2-PrimerSet2 | − | Negative infection |
| HostBiomarker | − | Negative infection |
| HostControl | + | Positive control |
| SARS2-PrimerSet1 | − | Negative infection |
| SARS2-PrimerSet2 | − | Negative infection |
| HostBiomarker | + | Positive non-COVID-19 infection or |
| | | pre-detection COVID-19 infection. |
| | | Recommend subject to re-test |
| HostControl | + | Positive control |

TABLE 8
M-RT-LAMP primers alignment with other coronaviruses

| Virus | GenBankID | % Mismatch |
|---|---|---|
| COVID-19 | MN908947 | 0 |
| Bat SARS-like CoV 2015 | MG772933.1 | 27.22 |
| Bat SARS-like CoV 2017 | MG772934.1 | 30.77 |
| Bat SARS CoV RM1/2004 | KY417144.1 | 40.83 |
| SARS CoV ZS-C | AY395003.1 | 42.60 |
| Civet SARS CoV SZ16/2003 | AY304488.1 | 39.64 |
| SARS CoV | NC_004718.3 | 40.24 |
| SARS CoV MA15 | FJ882957.1 | 40.24 |
| Middle East Respiratory CoV | NC_019843.3 | 52.07 |
| Belacoronavirus England 1 | NC_038294.1 | 54.44 |
| Murine hepatitis virus | NC_001846.1 | 53.25 |
| Human Coronavirus 229E | NC_002645.1 | 51.48 |
| Human Coronavirus NL63 | NC_005831.2 | 54.44 |
| Human Coronavirus HKU1 | NC_006577.2 | 53.25 |
| Human Coronavirus OC43 | NC_006213.1 | 49.11 |

TABLE 9
M-RT-LAMP Assay Application to Additional Organisms

| Other high priority | |
|---|---|
| pathogens from the | High priority organisms likely in |
| same genetic family | circulating areas |
| Human coronavirus 229E | Adenovirus (e.g. C1 Ad. 71) |
| Human coronavirus OC43 | Human Metapneumovirus (hMPV) |
| Human coronavirus HKU1 | Parainfluenza virus 1-4 |
| Human coronavirus NL63 | Influenza A & B |
| SARS-coronavirus | Enterovirus (e.g. EV68) |
| MERS-coronavirus | Respiratory syncytial virus |
| | Rhinovirus |
| | Chlamydia pneumoniae |
| | Haemophilus influenzae |
| | Legionella pneumophila |
| | Mycobacterium tuberculosis |
| | Streptococcus pneumoniae |
| | Streptococcus pyogenes |
| | Bordetella pertussis |
| | Mycoplasma pneumoniae |
| | Pneumocystis jirovecii (PJP) |
| | Pooled human nasal wash - to represent |
| | diverse microbial flora in the human |
| | respiratory tract |
| | Candida albicans |
| | Pseudomonas aeruginosa |
| | Staphylococcus epidermis |
| | Staphylococcus salivarius |

TABLE 10
Exemplary primers for host RNA biomarkers of infection.

| Gene | F3 Primer (5′-3′) |
|---|---|
| IFIT2 | CTGCCGAACAGCTGAGAATT (SEQ ID NO. 27) |
| IFIH1 | TCAGAAAGCAATGCAGAGA (SEQ ID NO. 28) |
| OAS2 | CTGAGTTCTGGCTCCACAC (SEQ ID NO. 29) |
| CXCL10 | CCAAGGTCTTTAGAAAAACTTGAA (SEQ ID NO. 30) |
| MxA | AGAGTGGCTGTGGGCAAT (SEQ ID NO. 31) |
| IFITM2 | AGCCTCCCAACTACGAGATG (SEQ ID NO. 32) |
| PARP12 | ACGCTGCCTTTCTACTTTGT (SEQ ID NO. 33) |
| IFIT3 | ACAGCAGAGACACAGAGGGCA (SEQ ID NO. 34) |
| MxB | GGAGGCACTGTCAGGAGT (SEQ ID NO. 35) |
| IRF9 | GCTGTCTGGAAGACTCGC (SEQ ID NO. 36) |
| RTP4 | GAGGGAAAAAATGGTTGTAGAT (SEQ ID NO. 37) |
| IFI27 | GCCACGGAATTAACCCGAGC (SEQ ID NO. 38) |
| Gene | B3 Primer (5′-3′) |
| IFIT2 | GCCAGTAGGTTGCACATTG (SEQ ID NO. 39) |
| IFIH1 | ACTTCCTTCTGCCAAACT (SEQ ID NO. 40) |
| OAS2 | CCCTTTGGCTTCAGTTTCCT (SEQ ID NO. 41) |
| CXCL10 | CTTGGAAGCACTGCATCG (SEQ ID NO. 42) |
| MxA | ACCAGATCAGGCTTCGTCA (SEQ ID NO. 43) |
| IFITM2 | TCACGTCGCCAACCATCT (SEQ ID NO. 44) |
| PARP12 | CCCCTTGCCGTAGGAAGT (SEQ ID NO. 45) |
| IFIT3 | TGTAGGCCAACAAGTIGT (SEQ ID NO. 46) |
| MxB | TGATGAGCTCATGGCTGATG (SEQ ID NO. 47) |
| IRF9 | CCTTCCTCTCAGAGGACACA (SEQ ID NO. 48) |
| RTP4 | CAACTTCGCTGGCAGGAGGAA (SEQ ID NO. 49) |
| IFI27 | CTGCTATGGAGGACGAGG (SEQ ID NO. 50) |
| Gene | FIP Primer (5′-3′) |
| IFIT2 | TCAAGTTCCAGGTGAAATGGCA-CTGCAACCATGAGTGAGAAC (SEQ ID NO. 51) |
| IFIH1 | CCAGATTTGGCTGAACTGTGGT-AGAATTTATCACAAGTTGATGGTC (SEQ ID NO. 52) |
| OAS2 | AACTCTCCTCCCGGGAGGTC-CAGCCCCGAGGTTTATGC (SEQ ID NO. 53) |
| CXCL10 | TCTCTTCTCACCCTTCTTTTTCATT-CCTGCAAGCCAATTTTGTC (SEQ ID NO. 54) |
| MxA | GACCACCACCAGGCTGATTGTC-GCCTGCTGACATTGGGTAT (SEQ ID NO. 55) |
| IFITM2 | GGATCACGGTGGACATCGGG-TCAAGGAGGAGCAGGAAGT (SEQ ID NO. 56) |
| PARP12 | TCCGTTCTGCTTCTGCATCTGT-AGTACAGAACCTGGCCCTC (SEQ ID NO. 57) |
| IFIT3 | GTTCCAGGTGAAATGGCATTTCAG-GTGAGGTCACCAAGAATTCC (SEQ ID NO. 58) |
| MxB | GCCTCACAGGGCTGCTTTTTCAAGAGGCAGCGGAATCGTA (SEQ ID NO. 59) |
| IRF9 | CAACATCCATGCGGCCCCTC-CTGCGCTGTGCACTCAAC (SEQ ID NO. 60) |
| RTP4 | CATCCAACTTCAGCGTCCATG-CTGGACTTGGGAGCAGACAT (SEQ ID NO. 61) |
| IFI27 | CAAAACTACGGCAGAGCCAG-TCTCACCTCATCAGCAGT (SEQ ID NO. 62) |
| Gene | BIP Primer (5′-3′) |
| IFIT2 | GGAGGGAGAAAACTCCTTGGAT-TGGCTTTGAATTCACGATTC (SEQ ID NO. 63) |
| IFIH1 | AGGTCTGGGGCATGGAGAAT-TGTGTCTGATTCTGAAACTACA (SEQ ID NO. 64) |
| OAS2 | ACCTGTTTCACAGTCCTGCAGC-GTGCTTCACCAGGCGAATT (SEQ ID NO. 65) |
| CXCL10 | GAATCCAGAATCGAAGGCCATCA-CCCTCTGGTTTTAAGGAGAT (SEQ ID NO. 66) |
| MxA | AATGTGGACATCGCCACCACA-AGATTCCGATGGTCCTGTCT (SEQ ID NO. 67) |
| IFITM2 | CCATGTGGTCTGGTCCCTGTTC-TTCACGGAGTACGCGAATG (SEQ ID NO. 68) |
| PARP12 | CGGCACCAGCGCCATTTTTG-CATGAACACCACAGACCCG (SEQ ID NO. 69) |
| IFIT3 | GACAGTGTCTCAAGGGATCTAGA-ATTGTAGCTTTGAACTCAGTG (SEQ ID NO. 70) |
| MxB | CAGCTACCGGAACACCGAGCGACGTTCTGGGCTTTGTGTA (SEQ ID NO. 71) |
| IRF9 | AGGTGTATCAGTTGCTGCCACC-TGCTGTCGCTTTGATGGTAC (SEQ ID NO. 72) |
| RTP4 | AACCTTCAGCTAGACTGCCT-AACCAGCCAAATGCTCTCTG (SEQ ID NO. 73) |
| IFI27 | TACAGTIGTGATTGGAGGAGTT-GATTCCCGCCGCAGTGAA (SEQ ID NO. 74) |
| Gene | LF Primer (5′-3′) |
| IFIT2 | GCCGTAGGCTGCTCTCCAAG (SEQ ID NO. 75) |
| IFIH1 | AAGAAGTTGCTCTTCCACTTGAG (SEQ ID NO. 76) |
| OAS2 | GGATTTATACAGATCAATGAGCCCT (SEQ ID NO. 77) |
| CXCL10 | |
| MxA | GGATGTACTTCTTGATGAGTGTCTT (SEQ ID NO. 78) |
| IFITM2 | AGCAGGGTTGTGGGGCA (SEQ ID NO. 79) |
| PARP12 | CCTTTTTGCCACTGGTAGACTTC (SEQ ID NO. 80) |
| IFIT3 | TGTGGAAGGATTTTCTCCA (SEQ ID NO. 81) |
| MxB | CACAGGCGACCACGACT (SEQ ID NO. 82) |
| IRF9 | GGAACCTCCTTAAATTCAGAACTCT (SEQ ID NO. 83) |
| RTP4 | GTTTTGCCTCTTGGATTAGTTCTTG (SEQ ID NO. 84) |
| IFI27 | GCCACCCTGACCACTTTGG (SEQ ID NO. 85) |
| Gene | LB Primer (5′-3′) |
| IFIT2 | GACAAAGTATTTTACCGGACTGAGT (SEQ ID NO. 86) |
| IFIH1 | |
| OAS2 | CTCCCGGCCCACCAAACTA (SEQ ID NO. 87) |
| CXCL10 | TACTGAAAGCAGTTAGCAAGGAAAG (SEQ ID NO. 88) |
| MxA | TCTCAGCATGGCCCAGGAGG (SEQ ID NO. 89) |
| IFITM2 | CACCCTCTTCATGAACACCTGCTG (SEQ ID NO. 90) |
| PARP12 | CGCCATCTGCCAGCAGAACTT (SEQ ID NO. 91) |
| IFIT3 | GATAGAGTGTGTAACCAGATTG (SEQ ID NO. 92) |
| MxB | TGGCCAGGTGGAGAAAGAG (SEQ ID NO. 93) |
| IRF9 | GCCAGCCAGGGACTCAGAA (SEQ ID NO. 94) |
| RTP4 | GGCTCAAGGGTGGAAGCAATA (SEQ ID NO. 95) |
| IFI27 | TGCTCAGTGCCATGGGC (SEQ ID NO. 96) |

TABLE 11
Exemplary pH indicators

| | | pH Transition | Low pH |
|---|---|---|---|
| Indicator | High pH Color | Range | Color |
| Bromocresol purple | Purple | 6.8-5.2 | Yellow |
| Neutral red | Clear-yellow | 8.0-6.8 | Red |
| Phenol red | Red | 8.2-6.8 | Yellow |
| Cresol red | Red | 8.8-7.2 | Yellow |
| Naphtholphthalein | Blue | 8.8-7.3 | Clear-red |
| m-Cresol purple | Purple | 9.0-7.4 | Yellow |
| Thymol blue | Blue | 9.6-8.0 | Yellow |
| Phenolphthalein | Red | 10-8.0 | Clear |

### REFERENCES

1. Batéjat C G, et al. (2021) Heat inactivation of the severe acute respiratory syndrome coronavirus 2 Journal of Biosafety and Biosecurity 3:1-3.
2. Bhadra S, et al. (2020) High-surety isothermal amplification and detection of SARS-CoV-2, including with crude enzymes bioRxiv.
3. Brewster C D, et al. (2018) LAMP assays of zika virus and other infectious agents will inevitably see expanded use due to their simplicity, sensitivity, specificity, and economy Annals of Translational Medicine 6:196.
4. Butler-Laporte G L, et al. (2021) Comparison of saliva and nasopharyngeal swab nucleic acid amplification testing for detection of SARS-CoV-2 JAMA Internal Medicine 181:353.
5. Cameron S J, et al. (2015) The human salivary microbiome exhibits temporal stability in bacterial diversity FEMS Microbiology Ecology 91:fiv091.
6. Chotiwan N, et al. (2017) Rapid and specific detection of Asian- and African-lineage zika viruses Science Translational Medicine 9:eaag0538.
7. Curtis K A, et al. (2018) A multiplexed RT-LAMP assay for detection of group M HIV-1 in plasma or whole blood Journal of Virological Methods 255:91-97.
8. Dao Thi V L, et al. (2020) A colorimetric RT-LAMP assay and LAMP-sequencing for detecting SARS-CoV-2 RNA in clinical samples Science Translational Medicine 12:eabc7075.
9. Davidi D, et al. (2020) Amplicon contamination in labs masquerades as COVID19 in surveillance tests medRxiv.
10. Flynn M J, et al. (2020) A simple direct RT-LAMP SARS-CoV-2 saliva diagnostic medRxiv.
11. Hardinge P, et al. (2019) Reduced false positives and improved reporting of Loop-Mediated isothermal amplification using quenched fluorescent primers Scientific Reports 9:7400.
12. Kellner M J, et al. (2020) (VCDI) VC-19 DI, pauli A, brennecke J. 2020. A rapid, highly sensitive and open-access SARS-CoV-2 detection assay for laboratory and home testing bioRxiv.
13. Khan P, et al. (2020) Isothermal SARS-CoV-2 diagnostics: tools for enabling distributed pandemic testing as a means of supporting safe reopenings ACS Synthetic Biology 9:2861-2880.
14. Kim DLee JYYang JSKim JWKim VNChang H (2020) The architecture of SARS-CoV-2 transcriptome Cell 181:914-921.
15. Lalli M A, et al. (2020) Rapid and extraction-free detection of SARS-CoV-2 from saliva with colorimetric LAMP medRxiv.
16. Lamb L E, et al. (2020) Rapid detection of novel coronavirus/Severe acute respiratory syndrome coronavirus 2 (SARS-CoV-2) by reverse transcription-loop-mediated isothermal amplification PLOS ONE 15:e0234682.
17. Mögling R, et al. (2020) Delayed laboratory response to COVID-19 caused by molecular diagnostic contamination Emerging Infectious Diseases 26:1944-1946.
18. Mori Y, et al. (2001) Detection of loop-mediated isothermal amplification reaction by turbidity derived from magnesium pyrophosphate formation Biochemical and Biophysical Research Communications 289:150-154.
19. Nagura-Ikeda M, et al. (2020) Clinical evaluation of Self-Collected saliva by quantitative reverse Transcription-PCR (RT-qPCR), Direct RT-qPCR, reverse Transcription-Loop-Mediated isothermal amplification, and a rapid antigen test to diagnose COVID-19 Journal of Clinical Microbiology 58:e01438-20.
20. Notomi T, (2000) Loop-mediated isothermal amplification of DNA Nucleic Acids Research 28:63e.
21. Rabe B A, et al. (2020) SARS-CoV-2 detection using isothermal amplification and a rapid, inexpensive protocol for sample inactivation and purification PNAS 117:24450-24458.
22. Ranoa D R E, et al. (2020) Saliva-Based molecular testing for SARS-CoV-2 that bypasses RNA extraction bioRxiv.
23. Seyrig G, et al. (2015) Selection of fluorescent DNA dyes for real-time LAMP with portable and simple optics Journal of Microbiological Methods 119:223-227. https://doi.org/10.1016/j.mimet.2015.11.004
24. Silva J, et al. (2021) Saliva viral load is a dynamic unifying correlate of COVID-19 severity and mortality medRxiv.
25. Taki K, et al. (2021) SARS-CoV-2 detection by fluorescence loop-mediated isothermal amplification with and without RNA extraction Journal of Infection and Chemotherapy 27:410-412.
26. Tang Y, Chen H, Diao Y (2016) Advanced uracil DNA glycosylase-supplemented real-time reverse transcription loop-mediated isothermal amplification (UDG-rRT-LAMP) method for universal and specific detection of tembusu virus Scientific Reports 6:27605.
27. Tanner N A, et al. (2015) Visual detection of isothermal nucleic acid amplification using pH-sensitive dyes BioTechniques 58:59-68.
28. Vogels C B F, et al. IMPACT Research Team (2020) SalivaDirect: a simplified and flexible platform to enhance SARS-CoV-2 testing capacity Med 2: e10.
29. Wright W F, Mackowiak P A (2020) Why temperature screening for coronavirus disease 2019 with noncontact infrared thermometers does not work Open Forum Infectious Diseases 8:ofaa603.
30. Wyllie A L, et al. (2020) Saliva or nasopharyngeal swab specimens for detection of SARS-CoV-2 New England Journal of Medicine 383:1283-1286.
31. Yang Q, et al. (2021) Just 2% of SARS-CoV-2-positive individuals carry 90% of the virus circulating in communities medRxiv: The Preprint Server for Health Sciences.
32. Yokota I, et al. (2020) Mass screening of asymptomatic persons for SARS-CoV-2 using saliva Clinical Infectious Diseases: An Official Publication of the Infectious Diseases Society of America 25:ciaa1388.
33. Zhang Y, et al. (2020) Enhancing colorimetric loop-mediated isothermal amplification speed and sensitivity with guanidine chloride BioTechniques 69:178-185.

1-12. (canceled)

13. A saliva stabilization solution comprising:
a quantity of reducing agent;
a quantity of a chelating agent, and
a quantity of one or more base solutions;
a quantity of a nuclease inhibitor; and
a nuclease-free water solution.

14. The solution of claim 13, wherein said reducing agent is selected from the group consisting of: tris(2-carboxylethyl)phosphine (TCEP), dithiothreitol (DDT), 2-mercaptoethanol, lithium aluminum hydride, Red-Al, Sodium amalgam, Sodium-lead alloy, Zinc amalgam Diborane; sodium borohydride (NaBH4), compounds containing the Fe2+ ion, iron(II) sulfate, compounds containing the Sn2+ ion, tin(II) chloride, Sulfur dioxide, Sulfite compounds, Dithionates, Thiosulfates, Iodides, Hydrazine, Diisobutylaluminium hydride (DIBAL-H), Oxalic acid, Formic acid, Ascorbic acid, Reducing sugars, Phosphites, phypophosphites, and phosphorous acid

15. The solution of claim 13, wherein said chelating agent is selected from the group consisting of: ethylenediaminetetraacetic acid (EDTA), egtazic acid, Chelation, 2,2′-Dipyridylamine, Acetylacetone, Alizarin, Alizarin Red S, Amidoxime, Amidoxime group, Aminoethylethanolamine, Aminomethylphosphonic acid, Aminopolycarboxylic acid, ATMP, Aza-crown ether, BAPTA, Bathocuproine, BDTH2, Benzotriazole, Benzoylacetone, Bidentate, BiPhePhos, Bipyridine, 2,2′-Bipyridine, 2,2′-Bipyrimidine, Bis(dicyclohexylphosphino)ethane, 1,2-Bis(dimethylarsino)benzene, 1,2-Bis(dimethylphosphino)ethane, 1,4-Bis(diphenylphosphino)butane, 1,2-Bis(diphenylphosphino)ethane, Calixarene, Carcerand, Catechol, Cavitand, Chelating resin, Chelex 100, Citrate, Citric acid, Clathrochelate, Corrole, Cryptand, 2.2.2-Cryptand, Cyclam, Cyclen, Cyclodextrin, Deferasirox, Deferiprone, Deferoxamine, Denticity, Dexrazoxane, Diacetyl monoxime, Trans-1,2-Diaminocyclohexane, 1,2-Diaminopropane, 1,5-Diaza-3,7-diphosphacyclooctanes, 1,4-Diazacycloheptane, 1,5-Diazacyclooctane, Dibenzoylmethane, Diethylenetriamine, Diglyme, 2,3-Dihydroxybenzoic acid, Dimercaprol, 2,3-Dimercapto-1-propanesulfonic acid, Dimercaptosuccinic acid, 1,1-Dimethylethylenediamine, 1,2-Dimethylethylenediamine, Dimethylglyoxime, DIOP, Diphenylethylenediamine, 1,5-Dithiacyclooctane, Domoic acid, DOTA, DOTA-TATE, DTPMP, EDDHA, EDDS, EDTMP, EGTA, Ethane-1,2-dithiol, Ethylenediamine, Ethylenediaminediacetic acid, Ethylenediaminetetraacetic acid, Etidronic acid, Fluo-4, Fura-2, Gallic acid, Gluconic acid, Glutamic acid, Glyoxal-bis(mesitylimine), Glyphosate, Hexaaza-18-crown-6, Hexafluoroacetylacetone, Hinokitiol, Homocitric acid, Hydroxyethylethylenediaminetriacetic acid, Iminodiacetic acid, Indo-1, Iron(tetraphenylporphyrinato) chloride, Isosaccharinic acid, Kainic acid, Lutetium (177Lu) oxodotreotide, MACHO catalyst, Malic acid, Metal acetylacetonates, Metal dithiolene complex, Metallacrown, Nickel bis(stilbenedithiolate), Nitrilotriacetic acid, Oxalic acid, Oxime, Pendetide, Penicillamine, Pentetic acid, Phanephos, Phenanthroline, O-Phenylenediamine, Phosphonate, Phthalocyanine, Phytochelatin, Picolinic acid, Polyaspartic acid, Porphine, Porphyrin, 3-Pyridylnicotinamide, 4-Pyridylnicotinamide, Pyrogallol, Salicylic acid, Sarcophagine, Sodium citrate, Sodium diethyldithiocarbamate, Sodium polyaspartate, Terpyridine, Tetramethylethylenediamine, Tetraphenylporphyrin, Tetrasodium EDTA, Thenoyltrifluoroacetone, Thioglycolic acid, Thujaplicine, TPEN, 1,4,7-Triazacyclononane, Tributyl phosphate, Tridentate, Triethylenetetramine, 1,1,1-Trifluoroacetylacetone, 1,4,7-Trimethyl-1,4,7-triazacyclononane, Triphos, Trisodium citrate, 1,4,7-Triethylcyclononane, and TTFA.

16. The solution of claim 13, wherein said base solutions is selected from the group consisting of: solutions containing NaOH, KOH, Mg(OH)2, Ca(OH)2, Al(OH)3, NH3, CaO. Sr(OH)2, Ba(OH)2, and Pyridine.

17. The solution of claim 13, wherein said saliva stabilization solution comprises a saliva stabilization solution:
wherein said quantity of reducing agent is TCEP;
wherein said quantity of chelating agent is EDTA, and
wherein said quantity of one or more base solutions NaOH;
wherein said quantity of nuclease inhibitor Proteinase K; and
wherein said nuclease-free base solution is nuclease-free water.

18. The solution of claim 17, wherein said saliva stabilization buffer comprises a 2× saliva stabilization solution:
wherein said quantity of TCEP is selected from: 5 mM, or 4-6 mM, or 143.3 mg, or 130-150 mg, 2.5 mM;
wherein said quantity of EDTA is selected from: 2 mM, 1-3 mM, 200 uL, 150-250 uL, and 1 mM;
wherein said quantity of NaOH is selected from: 29 mM, 25-34 mM, 290 uL, 240-340 uL, 14.5 mM, 50 ug/mL; and
wherein said quantity of Proteinase K is selected from: 100 ug/mL, 50-150 ug/mL, 10 mg, 5-15 mg.

19. (canceled)

20. The solution of claim 17,
wherein said quantity of nuclease-free water is added up to 100 ml.

21-23. (canceled)

24. The solution of claim 17, and further comprising wherein the solution is used in a nucleic acid amplification reaction.

25. The solution of claim 24, wherein said a nucleic acid amplification reaction is selected from the group consisting of: a strand-displacement amplification (SDA) reaction, a helicase-dependent amplification (HDA) reaction, a loop-mediated isothermal amplification (LAMP) reaction, and a polymerase-chain reaction (PCR) reaction, a nucleic acid amplification reaction to amplify and identify nucleic acids of a pathogen.

26. The solution of claim 24, wherein said nucleic acid amplification reaction uses at least one pH indicator dye.

27. The solution of claim 26, wherein said pH indicator dye is selected from the group consisting of: bromocresol purple, neutral red, phenol red, cresol red, naphtholphthalein, m-cresol purple, thymol blue, and phenolphthalein.

28. The solution of claim 17, wherein said saliva stabilization buffer is combined with a saliva sample at a 1:1 ratio.

29-50. (canceled)

51. A saliva stabilization solution comprising:
a quantity of reducing agent;
a quantity of a chelating agent, and
a quantity of one or more base solutions;
a quantity of a nuclease inhibitor;
a nuclease-free water solution; and
wherein said saliva stabilization solution is combined with a saliva sample from a subject in a test having a pH-dependent readout.

52. The solution of claim 51, wherein said reducing agent is selected from the group consisting of: tris(2-carboxylethyl)phosphine (TCEP), dithiothreitol (DDT), 2-mercaptoethanol, lithium aluminum hydride, Red-Al, Sodium amalgam, Sodium-lead alloy, Zinc amalgam Diborane; sodium borohydride (NaBH4), compounds containing the Fe2+ ion, iron (II) sulfate, compounds containing the Sn2+ ion, tin (II) chloride, Sulfur dioxide, Sulfite compounds, Dithionates, Thiosulfates, iodides, Hydrazine, Diisobutylaluminium hydride (DIBAL-H), Oxalic acid, Formic acid, Ascorbic acid, Reducing sugars, Phosphites, phypophosphites, and phosphorous acid

53. The solution of claim 51, wherein said chelating agent is selected from the group consisting of: ethylenediaminetetraacetic acid (EDTA), egtazic acid, Chelation, 2,2′-Dipyridylamine, Acetylacetone, Alizarin, Alizarin Red S, Amidoxime, Amidoxime group, Aminoethylethanolamine, Aminomethylphosphonic acid, Aminopolycarboxylic acid, ATMP, Aza-crown ether, BAPTA, Bathocuproine, BDTH2, Benzotriazole, Benzoylacetone, Bidentate, BiPhePhos, Bipyridine, 2,2′-Bipyridine, 2,2′-Bipyrimidine, Bis(dicyclohexylphosphino)ethane, 1,2-Bis(dimethylarsino)benzene, 1,2-Bis(dimethylphosphino)ethane, 1,4-Bis(diphenylphosphino)butane, 1,2-Bis(diphenylphosphino)ethane, Calixarene, Carcerand, Catechol, Cavitand, Chelating resin, Chelex 100, Citrate, Citric acid, Clathrochelate, Corrole, Cryptand, 2.2.2-Cryptand, Cyclam, Cyclen, Cyclodextrin, Deferasirox, Deferiprone, Deferoxamine, Denticity, Dexrazoxane, Diacetyl monoxime, Trans-1,2-Diaminocyclohexane, 1,2-Diaminopropane, 1,5-Diaza-3,7-diphosphacyclooctanes, 1,4-Diazacycloheptane, 1,5-Diazacyclooctane, Dibenzoylmethane, Diethylenetriamine, Diglyme, 2,3-Dihydroxybenzoic acid, Dimercaprol, 2,3-Dimercapto-1-propanesulfonic acid, Dimercaptosuccinic acid, 1,1-Dimethylethylenediamine, 1,2-Dimethylethylenediamine, Dimethylglyoxime, DIOP, Diphenylethylenediamine, 1,5-Dithiacyclooctane, Domoic acid, DOTA, DOTA-TATE, DTPMP, EDDHA, EDDS, EDTMP, EGTA, Ethane-1,2-dithiol, Ethylenediamine, Ethylenediaminediacetic acid, Ethylenediaminetetraacetic acid, Etidronic acid, Fluo-4, Fura-2, Gallic acid, Gluconic acid, Glutamic acid, Glyoxal-bis(mesitylimine), Glyphosate, Hexaaza-18-crown-6, Hexafluoroacetylacetone, Hinokitiol, Homocitric acid, Hydroxyethylethylenediaminetriacetic acid, Iminodiacetic acid, Indo-1, Iron(tetraphenylporphyrinato) chloride, Isosaccharinic acid, Kainic acid, Lutetium (177Lu) oxodotreotide, MACHO catalyst, Malic acid, Metal acetylacetonates, Metal dithiolene complex, Metallacrown, Nickel bis(stilbenedithiolate), Nitrilotriacetic acid, Oxalic acid, Oxime, Pendetide, Penicillamine, Pentetic acid, Phanephos, Phenanthroline, O-Phenylenediamine, Phosphonate, Phthalocyanine, Phytochelatin, Picolinic acid, Polyaspartic acid, Porphine, Porphyrin, 3-Pyridylnicotinamide, 4-Pyridylnicotinamide, Pyrogallol, Salicylic acid, Sarcophagine, Sodium citrate, Sodium diethyldithiocarbamate, Sodium polyaspartate, Terpyridine, Tetramethylethylenediamine, Tetraphenylporphyrin, Tetrasodium EDTA, Thenoyltrifluoroacetone, Thioglycolic acid, Thujaplicine, TPEN, 1,4,7-Triazacyclononane, Tributyl phosphate, Tridentate, Triethylenetetramine, 1,1,1-Trifluoroacetylacetone, 1,4,7-Trimethyl-1,4,7-triazacyclononane, Triphos, Trisodium citrate, 1,4,7-Triethylcyclononane, and TTFA.

54. The solution of claim 51, wherein said base solutions is selected from the group consisting of: solutions containing NaOH, KOH, Mg(OH)2, Ca(OH)2, Al(OH)3, NH3, CaO. Sr(OH)2, Ba(OH)2, and Pyridine.

55. The solution of claim 51, wherein said saliva stabilization solution comprises a saliva stabilization solution:
wherein said quantity of reducing agent is TCEP;
wherein said quantity of chelating agent is EDTA, and
wherein said quantity of one or more base solutions NaOH;
wherein said quantity of nuclease inhibitor Proteinase K; and
wherein said nuclease-free base solution is nuclease-free water.

56. The solution of claim 55, wherein said saliva stabilization buffer comprises a 2× saliva stabilization solution:
wherein said quantity of TCEP is selected from: 5 mM, or 4-6 mM, or 143.3 mg, or 130-150 mg, 2.5 mM;
wherein said quantity of EDTA is selected from: 2 mM, 1-3 mM, 200 uL, 150-250 uL, and 1 mM;
wherein said quantity of NaOH is selected from: 29 mM, 25-34 mM, 290 uL, 240-340 uL, 14.5 mM, 50 ug/mL; and
wherein said quantity of Proteinase K is selected from: 100 ug/mL, 50-150 ug/mL, 10 mg, 5-15 mg.

57. (canceled)

58. The solution of claim 55,
wherein said quantity of nuclease-free water is added up to 100 ml.

59-61. (canceled)

62. The solution of claim 55, wherein said test having a pH-dependent readout comprises a test having a pH-dependent readout selected from the group consisting of: a strand-displacement amplification (SDA) reaction, a helicase-dependent amplification (HDA) reaction, a loop-mediated isothermal amplification (LAMP) reaction, and a polymerase-chain reaction (PCR) reaction.

63. The solution of claim 62, wherein said a nucleic acid amplification reaction is selected from the group consisting of: a strand-displacement amplification (SDA) reaction, a helicase-dependent amplification (HDA) reaction, a loop-mediated isothermal amplification (LAMP) reaction, and a polymerase-chain reaction (PCR) reaction, a nucleic acid amplification reaction to amplify and identify nucleic acids of a pathogen.

64. The solution of any claim 63, wherein said nucleic acid amplification reaction uses at least one pH indicator dye.

65. The solution of claim 64, wherein said pH indicator dye is selected from the group consisting of: bromocresol purple, neutral red, phenol red, cresol red, naphtholphthalein, m-cresol purple, thymol blue, and phenolphthalein.

66. The solution of claim 55, wherein said saliva stabilization buffer is combined with a saliva sample at a 1:1 ratio.
